(19) United States
(12) Patent Application Publication
(10) Pub. No.: US 2003/0129659 A1
(43) Pub. Date: Jul. 10, 2003

(54) Library screening

(76) Inventors: E. Fayelle Whelihan, South Boston, MA (US); Robert C. Ladner, Ijamsville, MD (US)

Correspondence Address: FISH & RICHARDSON PC, 225 FRANKLIN ST, BOSTON, MA, 02110, US

(21) Appl. No.: 10/309,391

(22) Filed: Dec. 3, 2002

Related U.S. Application Data
(60) Provisional application No. 60/337,482, filed on Dec. 3, 2001.
(60) Provisional application No. 60/336,672, filed on Dec. 5, 2001.

(51) Int. Cl. G01N 33/53; G06F 19/00; G01N 33/48; G01N 33/50

(52) U.S. Cl. 435/7.1; 702/19

(57) ABSTRACT

Systems, methods, and apparati for screening libraries, particularly display libraries are disclosed. Methods can be automated or at least partially machine-based. Also disclosed are software and databases that interface with a library screening process such as a display library screening process. A computer system can be used to store, manage, and generate information that includes assay results and sample tracking from various automation stations. The system can include interfaces for project management, data analysis, and sample tracking and auditing. A database can manage hits identified during screening of a library. The database can be a relational database that includes tables for projects, libraries, screens, and hits.

### CROSS REFERENCE TO RELATED APPLICATIONS

This application claims priority to U.S. Provisional Applications Nos. 60/337,482, filed Dec. 3, 2001, and 60/336,672, filed Dec. 5, 2001, the contents of which are hereby incorporated by reference in their entirety.

### BACKGROUND

This invention relates to library screening. Recombinant techniques have allowed the discovery of artificial and natural polypeptides that have broad applications in the development of therapeutics, diagnostic agents (e.g., for imaging or binding assays), enzymes, and agents for affinity separations. One such recombinant technique is the construction of nucleic acid libraries that include diverse sequence content. Libraries can be screened by hybridization, genetic complementation, and polypeptide expression, among other activities. One challenge for screening expression libraries is to sort through large numbers of false positives to identify the best true positives that meet the screening criterion.

One type of expression library is a display library in which the expressed polypeptides are accessible for analysis and linked to the respective nucleic acids which encode them. One exemplary display library format uses filamentous bacteriophage. Polypeptides are fused to the protein coat of the phage and the nucleic acids that encode the polypeptides are located in nucleic acid encapsulated by the coat. Another display format uses cells. Polypeptides are expressed on the surface of cells and the nucleic acids encoding them are located within the cell. An exemplary application of a display library is the identification of library members that have a particular binding activity.

### SUMMARY

The invention provides systems, methods, and apparati for screening libraries, particularly display libraries. Many of the methods are automated or at least partially machine-based. The invention also provides software and databases that interface with the display library screening process. Of course, features of the invention can be used for other libraries, such as expression libraries and chemical libraries.

Information Management for Library Screening

The invention provides a computer system that stores, manages, and in some cases generates information that includes assay results and sample tracking from various automation stations. The system can include interfaces for project management, data analysis, and sample tracking and auditing.

Also provided is a database for managing hits identified during screening of a display library. The database can be a relational database that includes tables for projects, libraries, screens, and hits. For example, the hit table can include records for: ELISA assays, phage binding results, cell density, polypeptide sequence, nucleotide sequence, and originating library.

Accordingly, in one aspect, the invention features a method (e.g., a machine-based method) that includes: storing information that comprises associations between (a) individual library members and (b) assay information about each of a plurality of the individual library members; and evaluating the stored information to identify a subset of the individual library members. The evaluating can include filtering the stored information to identify a subset of library members. The library member can be a display library member, or a member of another library (e.g., an expression library, or a chemical library). The individual library members can also include members of different types of libraries.

The assay information can include one or more items entries for each of a plurality of the library members. The information can include data for a functional assay or a structural assay. Information can include, for example, one or more values from a qualitative or quantitative evaluation of a property associated with a library member, or even a state, e.g., “assay not performed” or “assay failed.” In one embodiment, the assay information includes information for a plurality of assays.

The method can further include receiving a query (e.g., a user) that comprises a criterion for functionality. The evaluating can include filtering the stored information to identify a library member for which the associated assay information (e.g., information including functional assay data) meets the criterion.

In another example, the evaluating can include identifying library members for which the assay data conforms to a statistically defined set. The statistically defined set can be a function of an average, median, mode, standard deviation, maximum, or minimum (e.g., selecting the best ten library members, etc.).

The method can further include storing associations between the (a) individual library members and biological sequence information. The evaluating can include identifying a subset for which the biological sequence information indicates a predetermined relationship with a reference sequence or sequences of other library members.

In one embodiment, the library members are selected from a library that comprises synthetic diversity, e.g., natural diversity, synthetic diversity, or both. At least some library members can be selected from a first library and at least some others can be selected from a second library. In another embodiment, at least some library members are selected from a composite library.

In an embodiment, the library members referenced by the information are identified by physical location of a clone of each respective library member and/or by biological sequence information.

In another case, the display library members include members that are isolated from a library for a first property and members that isolated from the library for a second property. For example, the first selection is for a first property and the second selection is for a second property that differs from the first property. In another example, the selections are for the same property. In still another example, the first property is binding to a first target and the second property is binding to a second target.

The assay information can include functional assay data. The functional assay data can include binding assay data and the criterion for functionality can be an activity, e.g., a binding activity or a catalytic activity. For example, the criterion is a preselected value, such as a minimum level of the activity or a maximum level of the activity. In another example the criterion is a range of levels of the activity. In one embodiment, the criterion describes a function of affinity or specificity.

In one embodiment, the activity is a binding activity. The binding activity can be represented as a value in proportion to affinity. In another embodiment, the binding activity is represented as a value in proportion to dissociation rate. The binding activity can be binding to a target or a non-target. Other examples of activities include an activity in a cellular assay, an enzymatic/catalytic activity, and in vivo activity in an organism.

Assay information can also include structural assay information, for example, information about a biophysical or structural assay (e.g., protein stability or folding).

The functional assay data can include, for each of a set of library members, binding activity for a first compound and binding activity for a second compound. For example, the first compound is a target compound and the second compound is a non-target compound.

The query can be received at a server from a client system. Information about the identified library member is sent from the server to the client system. The query can be received at the server from the client system across a network. At least some of the stored information can be received in electronic format from an apparatus, e.g., a sequencing apparatus or an assay apparatus. The information can be received across a network (e.g., an intranet or internet).

The method can include receiving information from an apparatus, e.g., in digital form (e.g., electronic, magnetic or optical form). The information can be received before the storing. Exemplary apparati include a nucleic acid sequencing apparatus, a plate scanner, and a liquid handling unit.

The method can further include displaying information to a user about the identified display library member, e.g., as text, a graph, hypertext or combinations thereof.

The method can further include sending information about the identified display library member to a client system. The sent information is, e.g., formatted, e.g., to include color information or graphical information. The format can be determined by user settings or preferences.

In one implementation, the stored information can include information for at least 10², 10⁴, or 10⁷ library members (e.g., display library members) that are isolated for binding to the same target.

Filtering can be used to identify a subset of library members, e.g., members for which the associated functional assay data meets the criterion.

The method can include instructing a sample handling instrument to manipulate clones corresponding to each of member of the subset. For example, the clones are distributed into separate containment areas, or into one or more common containment areas.

The method can also include hit-picking. For example, each of the display library members is stored at a unique address of one or more first arrays, and the method further includes instructing a sample handling instrument to transfer each member of the subset (or at least some, or all of the members of the subset) to a preselected address of one or more second arrays such that the order or total number of addresses for stored library members differs from the order or total number of addresses of the first array. The preselected address can be a unique address for each transferred member. The array can be a multi-sample carrier, e.g., a multi-well plate, a device that includes microfluidic channels, or a continuous array. The instructing can be, e.g., automatic, user-initiated, or triggered by a user preference. The method can include instructing an apparatus to evaluate each library member of the subset. The method can further include instructing a nucleic acid sequence instrument to sequence nucleic acid of each library member of the subset. The method can include designing a secondary library using biological sequence information for each member of the subset.

The method can further include producing a protein corresponding to a member of the subset. The method can also further include formulating the protein as a pharmaceutical composition, and optionally administering the pharmaceutical composition to a subject.

The method can include other features described herein. The invention also features a system that can effect one or more machine-based aspects of the method and an article of machine readable medium having instructions encoded thereon, the instructions causing a processor to a effect the method:

In another aspect, the invention features a method that includes providing a library that comprises a plurality of members, each member of the plurality including a diverse protein component and a nucleic acid that encodes the diverse protein component; selecting a set of members from the library; evaluating the set of the members to obtain a functional parameter for respective members of the set; sending information about the functional parameter for respective members of the set to a computer server for storage; querying the server with a criterion for functionality that can be used to identify a subset of the set that are characterized by functional parameter that satisfies the criterion; and filtering the stored information to identify a subset of library members for which the associated functional assay data meets the criterion. The subset can include a single member or multiple members. The library can include members that are not part of the plurality of members. For example, the library can include some members that do not include a diverse protein component, e.g., due to defective assembly.

The selecting of the set of display library members can include contacting the display library members to a target; and separating members that bind to the target from other members that do not bind to the target. In one embodiment, the target is immobilized during the separating.

The method can further include producing a protein corresponding to the diverse protein component of a member of the identified subset. The method can also further include formulating the protein as a pharmaceutical composition, and optionally administering the pharmaceutical composition to a subject.

The method can further include producing a nucleic acid which encodes variants of a protein corresponding to the diverse protein component of a member of the identified subset.

In another aspect, the invention features a machine-accessible medium which includes: data representing (a) information about selections to identify a polypeptide having a given property, (b) identifiers for library members that each encode a polypeptide, (c) results of assays for at least some of the library members; and, optionally, (d) biopolymer sequences for at least some of the display library members; and associations that relate one or more of: (1) the screen information and the library member identifiers; (2) library member identifiers and functional assay results (e.g., binding, catalytic, or biological assay results); and (3) library member identifiers and biopolymer sequences. The encoded data and associations can enable identification of display library members that satisfy a criterion, e.g., for comparison of assay information to a threshold value or sequence similarity to a query biopolymer sequence. The assay information can be about a functional assay, e.g., information about a binding activity, an activity in a cellular assay, an enzymatic/catalytic activity, in vivo activity in an organism. The assay information can be about an activity in a biophysical or structural assay (e.g., protein stability or folding).

The information about selections can include information about one or more of: a target, a selection condition, and a library.

The data can further represent information about projects, each project being associated with information about one or more selections. Instances of the information about each of at least some of the projects can be further associated with instances of information about a client.

The data representing biopolymer sequences can include data representing nucleic acid sequences and/or polypeptide sequences. In one embodiment, the data representing biopolymer sequences is parsed, e.g., trimmed (e.g., of at least some vector and/or invariant sequences). The data can include information indicating positions with data representing biopolymer sequences that are varied. For example, sub-fields can be used to indicate information about varied positions. The sequences corresponding to interaction regions can be indexed or otherwise indicated. For immunoglobulin sequences, for example, the sequences corresponding to CDR or CDR-coding regions, or FR or FR-coding regions can be indicated. The medium can further include quality information about the data representing biopolymer sequences.

The medium can include data representing information about selections and associations that related the selection information with screen

The medium can further include data representing information about clients and billing, instances of the information being associated with instances of the data representing projects.

System for Automation Work Flow

The invention also provides server interfaced with various automation workstations. The server can receive information from each workstation. The information can include sample tracking and experimental data. In some embodiments, the server can send instructions to a workstation. Workstations can include a robotic device that is used to manipulate a master plate of hits, a device for sequencing, a device for ELISAs, and so forth. The system can also monitor reagent use.

Accordingly, in one aspect, the invention provides a system that includes (1) a nucleic acid sequencing instrument that is configured to determine the nucleic acid sequence of library members selected by a library screen (e.g., a display library or an expression library); (2) an assay apparatus that is configured to assess a functional property of the selected library members; and (3) a server comprising, (a) a communication interface that receives information about the determined nucleic acid sequence of each of the selected library members from the nucleic acid sequence instrument and information about the assessed functional property for each of the selected library members from the assay apparatus, (b) a memory that stores the received information in association with information about the library screen, and (c) a processor that filters the received information to identify a subset of the select library members. The communication interface can receive information about separate sequence reads for each of the selected library members and the processor can compare information about one read of the reads to information about another of the reads.

The system can further include a (4) storage unit adapted to store multi-sample carriers, e.g., multi-chambered receptacles such as microtitre plates. The system can also include a sample picking apparatus, e.g., an apparatus configured to dispose picked samples on addresses of a multi-sample carrier. The sample picking apparatus can include a detector that detects an identifier on the multi-sample carrier. The sample picking apparatus can be interfaced with the server to communicate information about the detected identifiers to the server.

The system can further include a conveyor configured to move the multi-sample carrier, e.g., from the sample picking apparatus to the storage unit and/or from the sample picking apparatus to the assay apparatus.

The system can further include a sample handling device that rearrays multi-sample carriers. The processor can be configured to send instructions to the sample handling device.

The server processor can be configured to generate a report, e.g., automatically or in response to a trigger. The report can include information about events handled by the assay apparatus or the nucleic acid sequencing instrument. For example, the report can include results of searching a database of biopolymer sequences with at least one of the determined nucleic acid sequences. Further, the report can be formatted by a user-defined style.

The server memory can store nucleic acid sequence information for display library members selected by a plurality of screens in association with information about each of the screens. The plurality of screens can include screens for binding to different target compounds.

In one embodiment, the system generates an automatic alert, e.g., triggered by one or more of: a deviation between expected progress for the display library screen and actual progress, a overrepresentation of a sequence or motif among the sequence for display library members from a plurality of screens, or an expected reagent shortage.

In a related embodiment, the system stores information about a synthetic compound library screen, and includes information that indicates the block synthesis or synthetic pathway for a particular compound.

In another aspect, the invention features a system that includes a server storing (i) information about the determined nucleic acid sequence of each of a plurality of selected library members (e.g., display library members) from the nucleic acid sequence instrument and information about the assessed functional property for each of the selected library members from the assay apparatus, and (ii) software configured to distribute information from the stored information to client systems. In one embodiment, the software is also configured to receive a query, filter the stored information to identify a subset of the selected library members, and distribute information about the subset of the selected library members. In one embodiment, the software is also configured to receive information from a laboratory instrument.

In another aspect, the invention features a method that includes: receiving (e.g., automatically), from a nucleic acid sequencer, information about the nucleic acid sequence of library members identified in one or more library screens; receiving (e.g., automatically), from an assay device, information about functionality of the library members; and storing the received information in association with identifiers for the library members and an identifier for the library screen. The library members can be members of a display library.

The assay device can detect a sample identifier on a multi-sample carrier (e.g., a multi-well plate) that includes samples of the library members and the information received from the assay device includes information about the sample identifier. The sample identifier can indicate a library selection or selection campaign from which the library members are isolated.

In one embodiment, the stored information is further associated with a project identifier, and the project identifier is associated with at least another library screen. The method can include filtering the stored information to identify library members that satisfy a criterion.

In another embodiment, the method can include generating a graphical display representing information about the identified library members.

In still another embodiment, the method can include one or more of: formulating a polypeptide (or peptide) encoded by at least one of the identified library members as a pharmaceutical composition, and administering the pharmaceutical composition to a subject, coupling a polypeptide (or peptide) encoded by at least one of the identified library members to a label, administering the labeled polypeptide to a subject, and coupling a polypeptide (or peptide) encoded by at least one of the identified library members to a solid support.

In another aspect, the invention features a method that includes receiving information representing a plurality of biological sequences, each sequence corresponding to a nucleic acid library member selected from a nucleic acid library(e.g., an expression library such as a display library); evaluating the information for each biological sequence of the plurality to determine the location of a sequence feature, if present, wherein the sequence feature is characteristic of at least 5, 10, 20, 40, 60, 80, or 90% of the members of the nucleic acid library; and storing, extracting, or displaying information for a subsequence from each biological sequence for which the sequence feature is identified, the subsequence being defined as a function of the position of the sequence feature.

In one example, each member of the library encodes a protein that includes an immunoglobulin variable domain, and the library includes at least 10 different sequence variants of the immunoglobulin variable domain. For example, the plurality of sequence features can include sequence features located in one or more of the following regions: signal sequence, FR1, CDR1, FR2, CDR2, FR3, CDR3, FR4 or a constant domain. In another example, each member of the library encodes a protein that includes a Kunitz domain, and the display library includes at least 10 different sequence variants of the Kunitz domain. In still another example, each member of the library encodes a protein that includes an amino acid sequence that includes a varied region of less than 50, 40, 30, or 20 amino acids, the varied region including at least 4, 8, 12, or 18 varied positions.

In one embodiment, the method can further include trimming sequences not in the subsequence or extracting the subsequence.

In another aspect, the invention features a method that includes: disposing library members (a nucleic acid library, e.g., expression library members, e.g., display library members) into a first set of multi-sample carriers, each library member being disposed at a unique address of one of the multi-sample carriers of the first set; amplifying each library member; determining an assessment for each library member with respect to a property (e.g., a functional property, such as a binding property, or a sequence property, such the sequence of the library member nucleic acid component or polypeptide); storing information about the assessments of the library members; and filtering the information to identify a subset of the library members. The method can further include sequencing a nucleic acid component of each library member of the subset, manipulating each library member of the subset into a second set of multi-sample carrier, and/or prior to the disposing, selecting the library members for binding to a target. A magnetic particle processor can be used for the selecting.

In still another aspect, the invention features a method that includes: accessing stored information that comprises event identifiers, at least some of the identifiers being associated with a first screening of a library (e.g., a nucleic acid library such as a display library or other expression library); receiving a request for an event identifier for an event that relates to a second screening of a library; and generating an event identifier unique among the event identifiers in the stored information. The method can further include labelling a multi-sample carrier with the generated event identifier, e.g., an optically scannable identifier. In one example, the first and second screenings are screenings of the different libraries, e.g., different display libraries to identify binders to different targets. In another example, they are screens of the same library, e.g., the same display library, e.g., to identify binders to against different targets.

In another aspect, the invention includes: receiving, from a first workstation, information about a first event associated with a first screening of a display library; receiving, from a second workstation, information about a second event associated with the first screening; and storing the information about the first event and information about the second event in association with an indication of the first screening or other information associated with the first screening. The method can further include labelling a multi-sample carrier with the unique event identifier. The unique event identifier is, for example, a function of a project identifier or a screening identifier.

The labeled multi-sample carrier can be optically identified, e.g., by a high contrast image-able label such as a bar code or dot code.

The method can further include tracking the multi-sample carrier. For example, information about a third event associated with a second screening of a display library can be received from the first workstation. The first and second screenings can be screens for unrelated targets.

In yet another aspect, the invention features a method (e.g., a machine based method, or partially machine based method) that includes: initializing a project identifier for a project; generating a unique container identifier that is associated with the project identifier for a first multi-well container of library members (e.g., expression library members, e.g., display library members); labelling the multi-well container with the unique container identifier; and (e.g., automatically) disposing individual library members into wells of the multiwell container. The method can include amplifying each library member in the multiwell container or assessing a functional property (e.g., a binding or catalytic property) of each library member

In another aspect, the invention features a method that includes: receiving information about a nucleic acid sequence of a library member that is isolated from a composite of at least two libraries, wherein each library of the composite is constructed using a different limited set of codons at at least one position; parsing the information about the nucleic acid sequence into codons; and identifying an originating library from the libraries of the composite on the basis of the codon of the nucleic acid sequence at the at least one position.

In still another aspect, the invention features a method that includes constructing a first library of nucleic acids wherein each member of the first library includes one of a first limited set of codons at at least one varying position; constructing a second library of nucleic acids wherein each member of the second library includes one of a second limited set of codons at at least one varying position; and pooling members of the first and second library to form a composite library. The first limited set can differ from the second limited set at at least one corresponding varying position. For example, the first limited set of codons can include less than two codons for each given amino acid. In some embodiments, the first limited set of codons consists of a set of codons that is not a quadrant of a codon table.

The constructing can include synthesizing an oligonucleotide that comprises a region that is at least partially randomized, wherein the nucleotides of the region are synthesized by the addition of a trinucleotide from a mixture of trinucleotides, e.g., a limited set of trinucleotides. The codon usage at at least one corresponding constant position can differs between nucleic acids of the first and second libraries.

The method can further include: identifying a member of the pool that encodes a polypeptide having a given functional property, determining the sequence of the polypeptide having the given functional property. or determining from the determined sequence if the polypeptide is from the first or second library (or another library, e.g., a third library). The determined sequence can be the nucleic acid sequence.

Real-Time Information Delivery

The invention also provides an information management system that is used to monitor hits and project completion for library screening, e.g., a contract library screening service. A client accesses the system and receives an up-to-date report or specific information on a library screening project. The information delivery system can be interfaced with the database described above. For example, the invention features a method of delivering project information to a client across a network. The method can include: authenticating a client access request; accessing stored information that includes validation data for members of a diversity library or a selected fraction thereof; and transmitting information that includes an evaluation of the stored information across a network.

The invention also features a user interface that enables a user to select a subset of library members and displays information about each member of the subset. For example, the interface receives a parameter from the user, the parameter determining the selection of the subset of the library members. The interface can query the user for the parameter, e.g., a biopolymer sequence attribute or a biopolymer sequence, or a criterion for functionality. The selected subset of library members can have sequence similarity to the biopolymer sequence or have the biopolymer sequence attribute.

The interface can enable the user to access a database comprises stored data representing (a) information about screens of libraries, (b) identifiers for the library members, (c) results of functional assays for at least some of the library members; (d) biopolymer sequences for at least some of the library members; and associations that relate (1) the screen information and the library member identifiers; and (2) library member identifiers and functional assay results. The library members can be, e.g., display library members or expression library members. The library members can be identified in different selections. The interface can enable the user to distribute the displayed information to other users.

In another aspect, the invention features a server that includes a processor and memory, wherein the processor is configured to: store information about display library selection campaigns and display library members identified in the campaigns, filter the stored information to identify selected library members that are identified in the subset of the display library selection campaigns and that satisfy a criterion; and send to a remote user information about the selected library members. The processor can also receive queries from the remote user for information about display library members that satisfy a criterion, authenticate a remote user for permission to access the stored information for a subset of the display library screens. Each of the screens can be associated with a client, and the remote user can be authenticated if the remote user is identified as the client.

As used herein, the terms “protein” and “polypeptide” are used interchangeably. Both terms also encompass short peptides, e.g., peptides of 3 to 25 amino acids, as well as, of course, larger peptides, and multi-chain polypeptides.

Many aspects of the innovations described herein are applicable to library screening generally, e.g., the screening of libraries other than display libraries, e.g., the screening of an expression library, e.g., a cDNA expression library for an activity or a cellular phenotype, a nucleic aptamer library for a catalytic nucleic acid, a chemical library such as combinatorial library or a drug compound library.

All citations, including citations to publications, patents, and patent applications, are incorporated herein by reference in their entirety. The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a flowchart of an exemplary process for screening a display library.

FIG. 2 is a schematic of an exemplary database for storing information about a display library screen.

FIG. 3 is a schematic of an exemplary system for screening a display library.

FIG. 4 is a schematic of an exemplary network for screening a display library.

FIG. 5 is a flowchart of an exemplary process for screening a display library.

FIG. 6 is a schematic of an exemplary database for storing information about a display library screen.

FIG. 7 is a schematic of an exemplary process for screening a display library.

FIG. 8 is a view of an exemplary interface for hit picking.

FIG. 9 is a schematic of an exemplary automated system for screening a display library.

FIG. 10 is a flowchart of an exemplary process for tracking multi-well plates and associated events.

FIG. 11 is a schematic of an exemplary network for screening a display library for an external client.

FIG. 12 is a schematic of an exemplary server system.

### DETAILED DESCRIPTION

Display libraries are screened using a process that includes machine-based information management. At least some aspects of the process are automated. The technical effect of the process and of other processes described herein is to increase through-put for screening libraries and to enable the rapid access and analysis of information about the library screens. For example, the system manages the identification of polypeptides from one or more display libraries to be screened for multiple projects, each having different criteria.

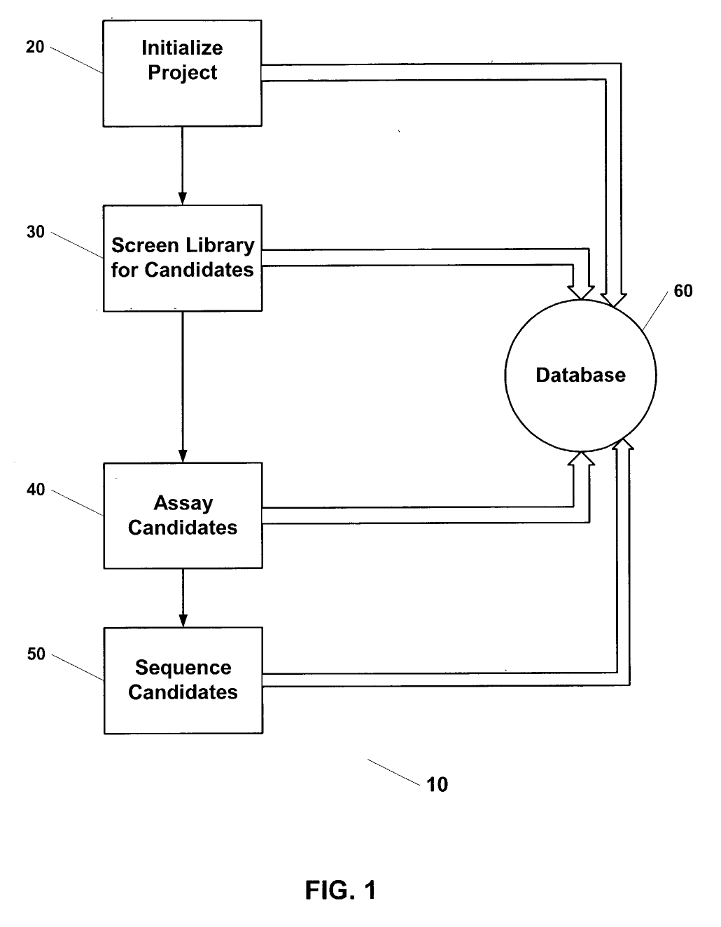

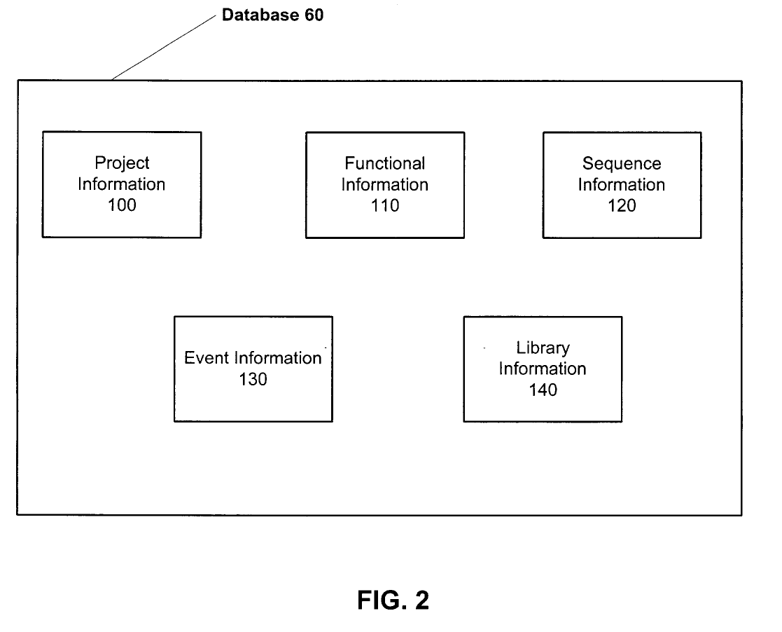

Referring to FIG. 1 and FIG. 2, the exemplary process 10 is used to screen a display library. Information from the process is collected through out the process in a relational database 60. The information can be accessed or analyzed at anytime.

The process 10 includes initializing 20 a so-called “project” that indicates, for example, the desired polypeptides that sought from the library. This information can be stored in a table of projects 100. Likewise, information about the library is stored in another table 140. Next, a display library is screened 30 to identify candidates for such desired polypeptides. Candidates are analyzed individually using high-throughput assays 40 that provide functional information 110 about the candidates. Candidates, or a subset of candidates, are sequenced 50. Sequence information 120 is stored in association with a record for each of the candidates.

In addition, the tracking and auditing of events associated with the process 10 generates event information 130 that is also stored in the database 60.

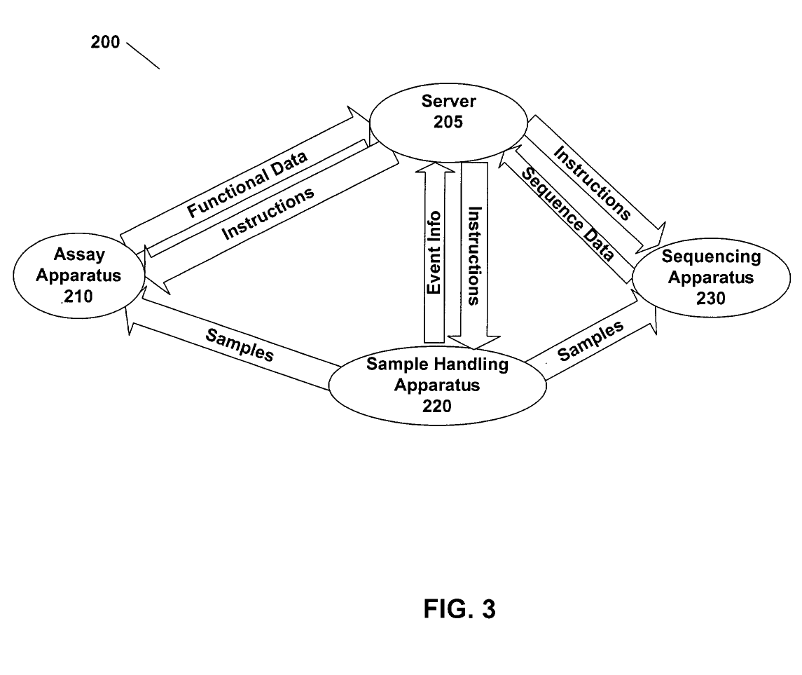

Referring to FIG. 3, the process 10 is implemented by an exemplary system 200. The system 200 includes a server 205, an assay apparatus 210, a sample handling apparatus 220, and a sequencing apparatus 230. The server 205 stores the database 60 information. The server 205 is interfaced with an assay apparatus 210 that generates data indicative of the functionality of display library members and sends it to the server 205. The server 205 is also in communication with a nucleic acid sequence apparatus 230. The apparatus 230 sends sequence data for individual library members to the server 205. In addition, the server 205 is in communication with a sample handling apparatus 220. The sample handling apparatus 220, as well as the assay apparatus 210, sends the server 205 information about events associated with sample handling. Of course, the system 200 can include additional apparati.

Instructions can be sent by the server 205 to any of the apparati 210, 220, 230. Such instructions might cause an apparatus to execute a particular method for particular candidates.

Further, within the system, the apparati 210, 220, 230 can also be physically connected. For example, the sample handling apparatus 220 can prepare a multi-well plate for assays. Although multi-well plates, such as 96- or 384-well microtitre plates, are referred to herein by way of example, other multi-sample carriers can be used for assays. Other examples of multi-sample carriers include multi-chambered carriers, a planar array (upon which samples are spotted at different addresses), “The Living Chip” (Biotrove, Inc., Cambridge Mass.), and devices with microfluidic channels. The assays are performed and the multiwell plates are automatically conveyed to the assay apparatus 210 to obtain a readout (See “Robotics,” below).

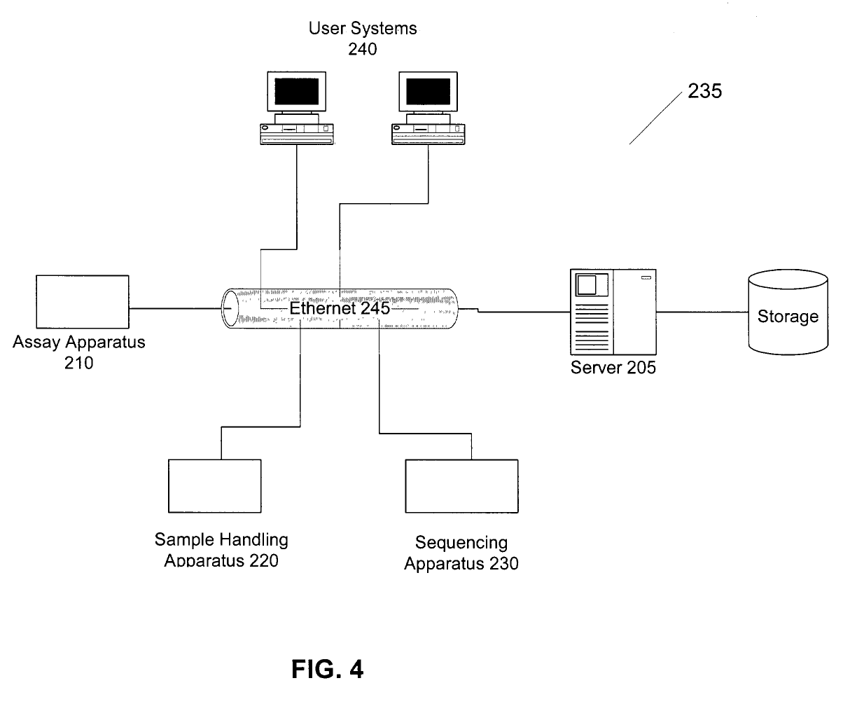

Referring to FIG. 4, the server 205 can be connected to a network, e.g., the Ethernet 245, that interfaces the server 205 with user systems 240, and the apparati 210, 220, 230. This network 235 enables the server 205 to efficiently exchange information with the apparati and enables users to access the information using interfaces on the user systems 240. The server 205 can be connected to a data storage unit 206, such as set of hard discs that are configured to redundantly store the database information 60.

Project Definition

The database 60 stores information about each project. Each project represents, for example, one or more screens to identify a polypeptide, e.g., one that fulfills an intend use. Thus, multiple projects can be conducted and managed simultaneously, and information from prior projects can inform current and future projects. A database table for a project can include the following fields:

Project_Name. The project can have a name for convenient reference by operators and for labeling reports, samples, and so forth.

Target. The target is the compound that is the basis of isolating members of the display library. In the case of where the intended use involves binding, the desired library members bind to the target. Of course, the target may be a target with respect to a functional activity, other than binding, or an activity which operates in addition to binding.

Intended Use. Options for intended uses can include: therapeutic, diagnostic, enzymatic, or purification. This field might be include a predefined list or may allow a free-text description, e.g., of variable length. See below for a discussion of intended uses.

Desired_Binding_Conditions. The desired binding conditions are based on the intended use. For example, for a therapeutic use, the desired binding conditions might be physiological strength buffers. For a purification use, the desired binding conditions might be the conditions of a buffer used in a prior purification step.

Desired_Release_Conditions. The desired release conditions are also based on the intended use. In the case of a therapeutic, the release conditions may be very stringent so that the desired polypeptide does not dissociate to a significant extent under physiological conditions. Such release conditions may be low pH, high pH, or chaotropic. For a purification use, the desired release conditions might be limited by the target compound, e.g., such that the target compound is not denatured or otherwise perturbed by elution from the desired polypeptide during purification.

Specificity_Requirement. This field can indicate the desired specificity for the candidate polypeptides. For some projects, the desired polypeptide binds to the target compound, but not a closely related compound, the non-target compound. If the target compound is a polypeptide, the related non-target compound can be an amino acid sequence homolog of the target, a conformational variant of the target, a proteolytic variant, or a glycosylated variant. An example of conformational variants are prion proteins. Likewise, this field can indicate catalytic specificity, e.g., the ability to catalyze one reactant but not a related, non-target reactant.

Other_Requirements. This field can include text or parameters that indicate other requirements that the candidate polypeptides have to meet. For example, with respect to therapeutic uses, such requirements can include antigenicity, clearance rate, half-life in circulation, and so forth. For enzymatic uses, such requirements can be kinetic parameters such as k_{cat}/Kₘ.

Client. The client can be the name of a party, e.g., a company or individual that requests an operator to execute the project. This field can also include a pointer to a client record in a table of clients within the relational database.

User. This field can be used to specific individuals who are associated with the project. These can include individuals with technical roles and/or managerial roles for the party hosting the project and similar individuals located externally at the client.

Authorization. This field can be used to assign permissions for the project. For example, permissions can be used to limit operators that can access information, enter (or upload) information, and/or execute events associated with the project. For example, individuals from a client may be given permission to access information about a particular project requested by the client, but not another project associated with another client, or even the same client.

Priority. This field can be used to assign a priority relative to other pending projects. Permission to modify the priority field can be limited to certain managers. The priority field can be used to allocate apparatus time, computational time (e.g., CPU time), and personnel time.

Milestones. The project record can include information about milestones for a project. These milestones can be dates in the future for which defined progress is forecast or dates in the past at which defined progress was attained. If appropriate the milestone field can include a pointer to information in another database table that is dedicated to milestone events.

Many other additional fields can be included.

Selections

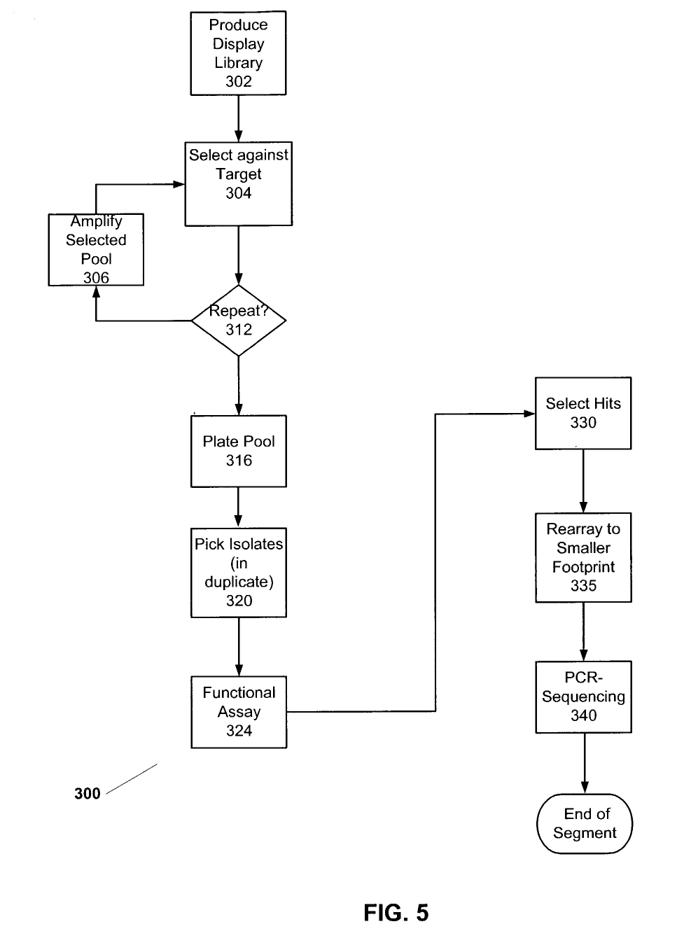

Referring to FIG. 5, a method for isolating a display library member includes providing (e.g., preparing) the display library 300. Exemplary methods for producing display libraries are described below (“Display Libraries”). Members are selected 304 from the library, e.g., by contacting the library to the target ligand and identifying members that bind to the target ligand. The target ligand can be immobilized on a solid support, or in solution, but later capturable. Unbound or weakly bound library members are washed from the support. Then, the bound library members are eluted from the support. Of course, other methods can also be used for selections. For example, the selection can be done in vivo to identify library members that bind to a target tissue or organ, e.g., as described in Kolonin et al. (2001) Current Opinion in Chemical Biology 5:308-313, Pasqualini and Ruoslahti (1996) Nature 380:364-366, and Paqualini et al. (2000) “In vivo Selection of Phage-Display Libraries” In Phage Display: A Laboratory Manual Ed. Barbas et al. Cold Spring Harbor Press 22.1-22.24. Selections can be used to select for enzymes, e.g., as described in Widersten et al. (2000) Methods Enzymol 328:389-404 (by binding to transition-state analog), Forrer et al. (1999) Current Opin. Struct. Biol 9:514-520, Gao et al. (1997) Proc. Natl. Acad. Sci. USA 94:11777, and Baca et al. (1997) Proc. Natl. Acad. Sci. USA 94:10063.

In some cases, non-specific binding and other non-ideal properties require more than one cycle of selection. Additional cycles of selection increase the enrichment for candidate library members. If repeating the selection step 314 is required, eluted library members can be amplified 306 then reapplied to the target ligand. Depending on the implementation, different numbers of cycles of selection may be sufficient to identify a pool of candidate library members from a library having a vast diversity. For example, one, or two rounds of selection may be sufficient. A set of cycles of selections is referred to as a selection campaign.

Since additional rounds of selection may increase bias in the selected library members and may result in the loss of members that are rare or are impaired relative to other members for reasons unrelated to their suitability as a candidate. For example, some library members may be impaired for replication in a host cell.

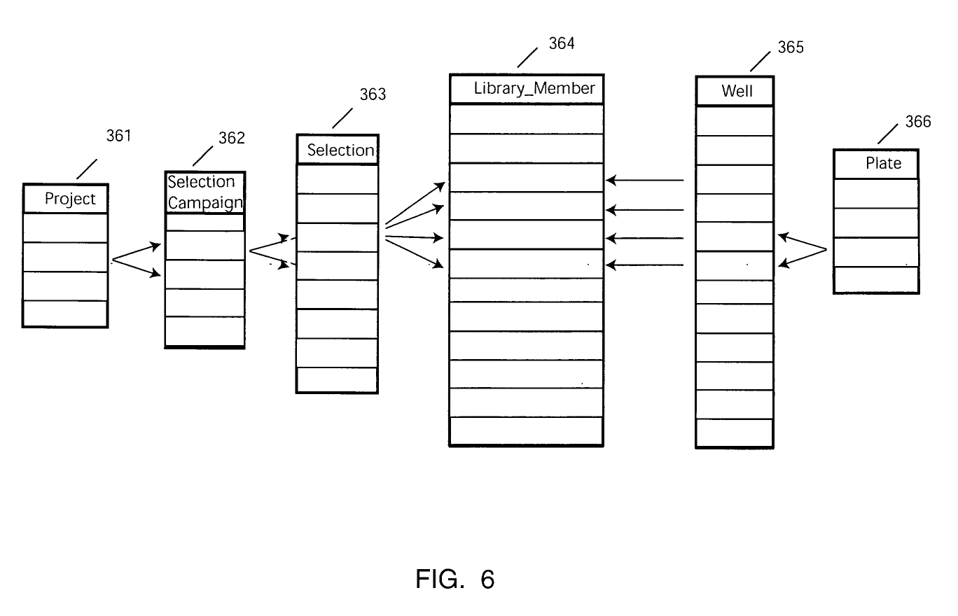

Referring to FIG. 6, the parameters for each of the selections are recorded in the database in a table for selections 363. The selections are associated with a particular selection campaign by an entry in a table for selection campaigns 362. As described, selection campaigns in turn are associated with a particular project in a table of projects 361.

Selection. A record for a selection can include the following fields:

Selection_Campaign. This field indicates the selection campaign for which the selection was performed.

Library_Source. This field indicates the source of display library members. The source can be a library preparation as is the case for the first selection of a selection campaign or the output of a previous selection as is the case for subsequent selections of a selection campaign.

Selection_Round. This field is an integer value that indicates the position of the selection within the selection campaign. For example, “1” indicates that the selection is the first selection, and so forth.

Input_Size. This parameter indicates the number of display library members contacted to the target.

Output_Size. This parameter indicates the number of display library members eluted from the target.

FOI. This is the fraction of the input display library members that are recovered by the selection campaign.

Target_Preparation. This can include a reference to a table of target preparations. For example, target compounds can be prepared in multiple batches, e.g., on different dates and/or using different processes. Such information is tracked and can be used to determine if identified ligands are correlated with particular preparations.

Selection_Campaign. A record for a selection campaign can include the following fields:

Project. This is a pointer to the project for which the selection campaign was initiated.

Library. This is a pointer to a library record that describes the display library used.

To identify all the selections associated with a selection campaign, the table of selections is queried for all entries that point to the selection campaign. Each selection entry includes an indication (Selection_Rank) that indicates its relative position in the selection campaign process.

A project can be associated with more than one selection campaign.

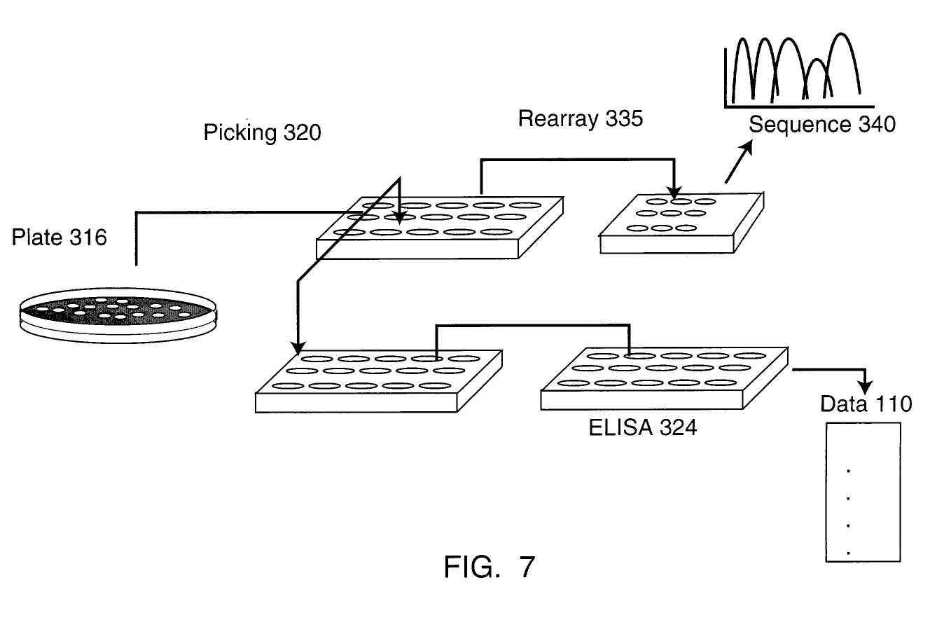

After selection, the identified members of the pool are individually isolated. Referring now to FIG. 5 and FIG. 7, for a phage display library, for example, the pool can be infected into bacterial cells which are then plated 316 at a density such that individual colonies or plaques are formed from each infection event. The individual colonies are picked 320 into wells of a multi-well plate, e.g., a 94- or 364-well plate, using an automated colony picker. Typically, the colonies are picked in duplicate, e.g., into corresponding wells of two identical plates. One of the plates is archived. The other can be used as a source for subsequent analyses.

Automated picking enables the picking of at least 100, 10³, 10⁴, 10⁵ (or more) selected library members. Each of these library members can then by analyzed individually as described below.

Referring again to FIG. 6, information about the picking is stored in the database 60, e.g., using cross-referenced records for, respectively, the plate 366, each well 365, and each display library member 364.

Multi_Well_Plate. A record for a multi-well plate can include:

Plate_ID. This field records the unique plate identifier which is automatically generated and labeled on the plate. See “Bar Coding & Event Auditing,” below.

Plate_Type. This field indicates the plate type, e.g., catalog number and manufacturer.

Plate_Size. This field indicates the number of wells on the plate, e.g., 96 or 364.

Storage_Location. This field indicates where the plate is physically stored. This can be a location in a freezer or in a plate hotel.

Project. This field indicates the project for which the plate was generated.

Date. This field indicates the date that the plate was entered into the system. Typically this is the date that display library members were disposed in wells of the plate.

Operator. This field indicates the person who directed or instructed entry of the plate into the system.

Well. A record for a well can include:

Plate_ID. This field is a pointer to the record for the multi-well plate for the well.

Well_X_Coordinate. This field indicates the x coordinate of the well on the plate.

Well_Y_Coordinate. This field indicates the y coordinate of the well on the plate.

Contents. This field indicates the sample disposed in the plate. It can be a pointer to a record for a display library member.

Library_Member. A record for a display library member can include:

Phage_ID. This field can include a unique identifier that identifies the library member in the database 60.

Selection_Campaign. This field includes a pointer that references the entry for the display library selection campaign from which the library member was isolated.

Plate_ID. This field include a pointer that references the entry for a multi-well plate in which the library member is stored.

Well_ID. This field includes a pointer that references the entry for the well in which the library member is stored.

Isolation_Date. This field indicates the date that the library member was isolated.

Operator. This field indicates the operator that oversaw the isolation of the library member.

AA_seq_ID. This is a pointer to a record that includes a string setting forth the amino acid sequence encoded by the display library member.

DNA_seq_ID. This is a pointer to a record that includes a string setting forth the nucleic acid sequence encoded by the display library member. A related record can provide a fingerprint, e.g., of a CDR or framework of an antibody.

SubLibrary_ID. In implementations in which a composite library is screened, this is a pointer to a record that documents a sublibrary that is determined to be the source of the display library member. The sublibrary is one of the component populations of the composite library.

Originating_Library. This is a pointer to a record that documents that library that was screened to identify the library member.

Assay_Results. This field can include a Boolean operator that indicates if a record of assay results is available in the table of functional information 110 for the display library member. In another implementation, this field can include pointers to one or more such records or can include the functional information itself.

A record for a display library member can be initialized and used before information for some of the fields is available. For example, nucleic acid and amino acid sequence information may only be associated with the record after the library member is assayed and approved for sequencing.

Assay

Referring to FIG. 5 and FIG. 7, the individual library members are analyzed using an assay 324, typically a high through-put assay. The assay determines functional information 110 for the polypeptide component being displayed for each library member. The functional information can be obtained for the polypeptide component when it is either attached or removed from the library vehicle, e.g., the bacteriophage. The functional information 110 is recorded in the database 60 in a table of assay results. Each entry in the table includes a field that points to the display library member being assayed and another field that stores the result of the assay, and other relevant information such as background levels, and results for controls.

The functional information 110 can relate to one or more of the following: a binding activity (including, for example, information related to specificity, a kinetic parameter, an equilibrium parameter, avidity, affinity, and so forth), a catalytic activity, a structural or biochemical property (e.g., thermal stability, oligomerization state, solubility and so forth), and a physiological property (e.g., renal clearance, toxicity, target tissue specificity, and so forth) and so forth. In some embodiments, a field within each record of a table of functional information indicates the property being assayed. In other embodiments, the functional information includes, e.g., multiple tables, each table for a different property or assay.

A variety of possible assays, including homogenous assays, are described below. For example, ELISAs can be used as an assay to identify functional information about binding. A database record for an ELISA assay can include the following information:

Target_Preparation (a pointer to a record for the target preparation); multi-well plate type; amount of target (e.g., in ng/well); blocking agent; blocking agent concentration; incubation time; incubation temperature; incubation buffer composition; incubation pH; incubation volume; wash buffer; number of washes; wash volumes; wash time; recognizer molecule (“RM”, e.g., the enzyme-linked probe, such as an antibody to a constant region of the display library members); amount of RM/well; time for RM binding; temperature for RM binding; wash buffer for RM; volume of RM washes; number of washes; developing agents; amount of developing agents; time for development; and expected signal range.

Assays for functional information are also discussed below (see, e.g., “Post-Processing”)

Hit Picking

The so-called “hit-picking” process 330 includes that analysis of functional information to identify individual library members that meet a given criterion. For example, the functional information can relate to the ability of the polypeptide encoded by each library member to bind to the target. In this case, a criteria for analysis may be a minimum binding activity. The database of functional information is filtered to identify the individual library members that meet the criteria.

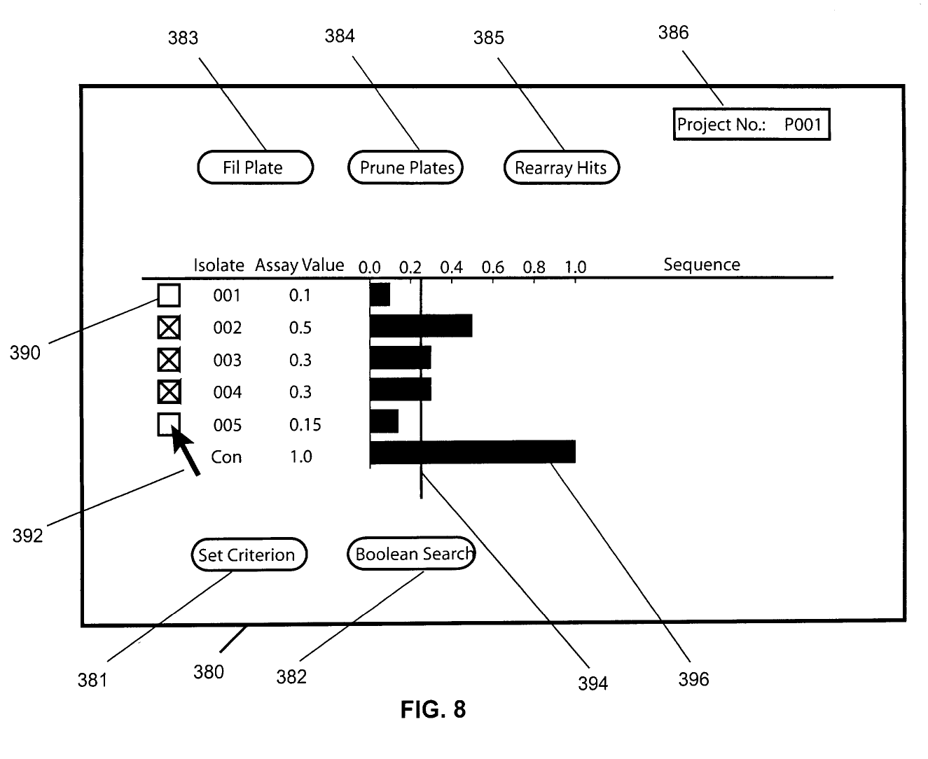

The server can include an interface for hit-picking. The server queries a user for a type of criterion, e.g., a particular assay or other requirement (e.g., particular sequence or library member). The server filters the database of functional information to identify library members of the screen (or of the project) that meet the criterion. Information for each identified library member can be displayed, or a summary of the results can be displayed (e.g., indicating the number of library members identified, the average score or median score). An example of a display of results for individual library members is depicted in FIG. 8.

The operator/user can approve the results or can alter the criterion in order to select more or fewer members. Further, Boolean search terms can be used to add (e.g., using an OR search) or to reduce the number of identified hits (e.g., using AND). This can be particularly useful if more than one functional assay has been performed. For example, this can be identified that bind to a target but which do not bind to a non-target (e.g., using AND NOT).

This information is then communicated to a sample handling unit which moves 325 individual clones from the multi-well plates as originally arrayed to a second set of multi-well plates (a so-called “re-arraying” process 325). Since only chosen library members are included in the second set, the second set is reduced in size relative to the initial set of multi-well plates. This reduced footprint conveniently facilitates downstream manipulations.

An exemplary hit picking interface 380 to the database 60 is depicted in FIG. 8. The interface enables the user to select display library members manually or automatically for a given project which can be indicated on the interface in the title bar 386. The interface displays identifiers for each library member (see column labeled “Isolate”) and its corresponding assay value in numerical (see column labeled “Assay Value”) and graphical format 396. If available, the sequence or a portion thereof can be shown for each library member. A control for the assay can also be graphed.

In one mode, the user selects to filter the library members using a criterion. This mode can be activated by triggering the “Set Criterion” button 381 and responding to a query that requests a property to set the criterion for. Typically, the property is one of the functional assay results. Next, the user is queried for a threshold value which can be indicated on the graphical display 396 by a so-called “cut-off” line 394. In some implementations the cut-off line 394 itself can be positioned by the user using the cursor 392 to indicate the threshold value.

The server 205 filters the display library members for members that meet the criterion. The checkbox 390 can be automatically selected for members that meet the criterion as depicted in example shown in FIG. 8 where the criterion is an assay value of at least 0.25. In another example, not shown here, the interface only displays library members that meet the criterion.

In another mode, the user can manually select, e.g., using a cursor 292 controlled by a mouse, one or more library members. The selection can be triggered by “checking” one of the checkboxes 390. The user can also select an option to be queried for the criterion or for a search expression (e.g., a Boolean search expression).

In still another mode, the user elects to query the library members using a Boolean search by selecting the checkbox 382. The user enters multiple search terms to filter the information for library members against. The interface then lists or otherwise indicates library members that meet the search terms.

The interface 380 also includes selectable boxes to fill 383 or prune 384 the list of selected library members. For example, the interface can display an indication of the number of selected library members that must be added or removed in order to produce an integral number of multi-well plates. This feature encourages the user to use every available well on a multi-well plate for rearraying. When the user has completed indicating selections, the “Rearray Hits” button 386 is selected. This can automatically deliver the rearraying instructions to the sample handling device

The interface can also display the library members as groups, e.g., using bars on a histogram to indicate the distribution of functionality among the assayed candidates. In this example, the user can select particular bars for further analysis, or a range of bars, e.g., by moving a cut-off line to truncate the histogram.

Of course, in some implementations, hit picking is not required. Selected library members can be retrieved from the containers into which the members were initially picked on an “as-needed” basis. In still other implementations, the re-arraying includes processing each selected library member. For example, a relevant insert of each selected library member can be subcloned or otherwise inserted into a different nucleic acid vector.

Sequencing

Referring to FIG. 5 and FIG. 7, the nucleic acid sequence of each library member of the second set is determined 340. For example, each member can be PCR amplified with primers that anneal to invariant regions of the library. The primers are positioned such that the sequenced region corresponds to a region that varies among the library members. The samples are amplified and sequenced using a PCR sequencing reaction.

The reactions are analyzed in a capillary sequencing device 230 such as the Applied Biosystems ABI3700. The ABI3700 can be programmed to automatically send sequencing results to the server 205 with information that associates each read with a display library member and information about the sequencing reaction, e.g., the primer used. Of course, such information can also be manually uploaded to the server 205 or transferred using a diskette or other related storage medium. Other sequencing methods can be used, e.g., “sequencing by hybridization” (see, e.g., U.S. Pat. Nos. 5,202,231, 5,695,940, and 6,007,987) and other nucleic acid array-based sequence determinations.

For quality control purposes, more than one read can be made for each region of sequence. For example, primers that anneal to complementary strands can be used to obtain a forward and reverse read of a given segment. Multiple reads can be analyzed using base-calling software such as PHRED and PHRAP (see, e.g., Ewing and Green (1998) Genome Research 8:175-185; Ewing and Green (1998) Genome Research 8:186-194; and Gordon et al. (1998) Genome Research. 8:195-202) to obtain a certainty value for each sequenced nucleotide.

The server 205 uses the certainty values to verify the nucleic acid sequence. If the certainty values fail preset thresholds, an alert is triggered that effects one or more of the following: automatically directing the sequencing apparatus 230 to resequence the display library member in question; notifying an operator, e.g., by email or an alert message box; and/or appending a flag to the nucleic acid sequence record that further verification is required.

For verified sequences in particular, the server 205 can automatically translate the nucleic acid in the relevant reading frame. The relevant reading frame can be indicated by the display libraries design or by inference. The database 60 can include a number of static tables that are used for translating the nucleic acid sequence. These tables include:

Amino_Acid_list: This table has a column for the names of the 20 amino acids and a column for their coded identifiers. Optionally, the table can include additional columns for the three-letter standard name (e.g., “Ala”), and the single-letter standard name (e.g., “A”); and

Codon Table: This table associates all 64 trinucleotides with the amino acids or stop codon that they encode.

The server 205 parses the nucleic acid sequence into codons and looks up in the codon table the amino acid that is encoded. The code for the amino acid, e.g., as provided by the amino acid table, is appended to a string in an amino acid sequence record in a table of amino acid sequences. If appropriate for the library in question, the server 205 also verifies that the amino acid sequence is consistent with the design of the library. For example, the amino acids are required to match a template in constant regions (e.g., framework regions for an antibody library and cysteines for a cysteine loop library) and must also match a set of allowed amino acids in variable regions. This verification, of course, can also be performed at the nucleic acid sequence level, e.g., as discussed for composite libraries below.

Some display libraries display multi-chain sequences, e.g., a protein that includes two polypeptide chains. For example, antibody Fab fragments include a heavy and a light polypeptide chain. The variant regions of both chains can be sequenced, or, in some implementations, it may suffice to sequence just one chain. For example, only one chain may include a variant region.

As one possible alternative to sequencing the complete variant regions of a library member, the library member can be fingerprinted by digestion with one or more restriction enzymes, or can be sequenced using a single dideoxy nucleotide to generate a tract, e.g., a T-tract. For some libraries, e.g., libraries that include an untranslated nucleic acid tag sequence, it may be sufficient to sequence a small region such as the tag rather than the complete variant regions. After additional winnowing of candidate display library members, partially sequenced or fingerprinted library members can be sequenced to determine the sequence of the complete variant regions, e.g., an entire domain or a segment that is varied such as a CDR.

Robotics

Various robotic devices are employed in the automation process. These include multi-well plate conveyance systems, magnetic bead particle processors, liquid handling units, colony picking units.

These devices can be built on custom specifications or purchased from commercial sources, such as Autogen (Framingham Mass.), Beckman Coulter (USA), Biorobotics (Woburn Mass.), Genetix (New Milton, Hampshire UK), Hamilton (Reno Nev.), Hudson (Springfield N.J.), Labsystems (Helsinki, Finland), Perkin Elmer Lifesciences (Wellseley Mass.), Packard Bioscience (Meriden Conn.), and Tecan (Mannedorf, Switzerland).

Each of these devices can have their own specialized data formats or can export data in a standard form, such as tab-delimited text. The server 205 can include scripts or other software that parses these data formats into information that can be processed and stored in the database. The server 205 can also be configured to communicate with each device using commands and other signals that are interpretable by the device. These customized interfaces can be routinely built from specifications provided by the device manufacturer.

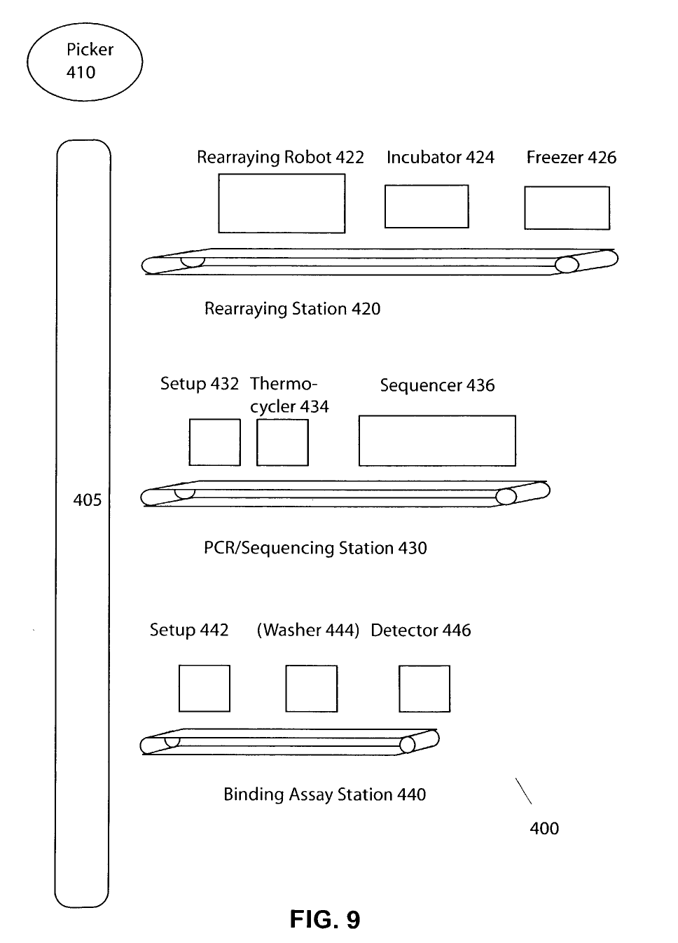

FIG. 9 depicts an exemplary automated system 400 for implementation of the process 10 and the system 200 schematized in FIG. 3. The system 400 includes conveyors 405 that transport multi-well plates between stations 420, 430, 440.

For example, the system can include a liquid handling station 420 that prepares multi-well plates for colony picking. This preparatory system fills the wells of the plates with sterile media. The plate is then robotically positioned in the plate picker 410. After picking in singlicate, duplicate (or higher), a pair of plates can be automatically transported to an incubator 424 for growth. After, one of the pair is moved to storage, e.g., a 4° C., −20° C. or −80° C. storage 420. The other is transported to the binding assay station 440.

In the example of an ELISA assay, the assay station 440 includes an automated liquid handling apparatus 442 that prepares ELISA plates, a washer 444 for washing ELISA plates, and a detector 446 for quantifying binding.

After the assay results are analyzed and hits are picked, the stored plate can be used to re-array the picked hits into a second pair of multi-well plates. Rearraying can be performed by the rearraying robot 422 located at the automated liquid handling station 420. After rearraying the pair of plates is transported to the incubator 424 for growth. One of the pair is automatically transported to storage 426, whereas the other is transported to a sequencing set up station 430 by the conveyor 405.

The sequencing setup station 430 prepares multi-well plates for the PCR sequencing reaction, e.g., in a thermal cycler 434, configured to accept multi-well plates. Each well of the plate is seeded with a sample of cells that include the display library member (e.g., phage infected cells in the case of a display library). After PCR amplification or DNA preparation and sequencing, the samples can be manually or automatically loaded onto a sequencing apparatus 436, such as the ABI3700.

Automated Selections

Referring again to FIG. 1, the screening process 30 can be performed manually or using an automated method. One example of an automated selection uses magnetic particles.

In this case, the target is immobilized on the magnetic particles, e.g., as described below. The KingFisher™ system, a magnetic particle processor from Thermo LabSystems (Helsinki, Finland), can be used to select display library members against the target. The display library is contacted to the magnetic particles in a tube. The beads and library are mixed. Then a magnetic pin, covered by a disposable sheath, retrieves the magnetic particles and transfers them to another tube that includes a wash solution. The particles are mixed with the was solution. In this manner, the magnetic particle processor can be used to serially transfer the magnetic particles to multiple tubes to wash non-specifically or weakly bound library members from the particles. After washing, the particles are transferred to a tube that includes an elution buffer to remove specifically and/or strongly bound library members from the particles. These eluted library members are then individually isolated for analysis as described above or pooled for an additional round of selection.

The use of automation to perform the selection increases the reproducibility of the selection process as well as the through-put.

An exemplary magnetically responsive particle is the Dynabead® available from Dynal Biotech (Oslo, Norway). Dynabeads® provide a spherical surface of uniform size, e.g., 2 μm, 4.5 μm, and 5.0 μm diameter. The beads include gamma Fe₂O₃ and Fe₃O₄ as magnetic material. The particles are superparamagnetic as they have magnetic properties in a magnetic field, but lack residual magnetism outside the field. The particles are available with a variety of surfaces, e.g., hydrophilic with a carboxylated surface and hydrophobic with a tosyl-activated surface. Particles can also be blocked with a blocking agent, such as BSA or casein to reduce non-specific binding and coupling of compounds other than the target to the particle.

The target is attached to the paramagnetic particle directly or indirectly. A variety of target molecules can be purchased in a form linked to paramagnetic particles. In one example, a target is chemically coupled to a particle that includes a reactive group, e.g., a crosslinker (e.g., N-hydroxy-succinimidyl ester) or a thiol.

In another example, the target is linked to the particle using a member of a specific binding pair. For example, the target can be coupled to biotin. The target is then bound to paramagnetic particles that are coated with streptavidin (e.g., M-270 and M-280 Streptavidin Dynaparticles® available from Dynal Biotech, Oslo, Norway). In one embodiment, the target is contacted to the sample prior to attachment of the target to the paramagnetic particles.

Another class of specific binding pair is a peptide epitope and the monoclonal antibody specific for it (see, e.g., Kolodziej and Young (1991) Methods Enz. 194:508-519 for general methods of providing an epitope tag). Exemplary epitope tags include HA (influenza hemagglutinin; Wilson et al. (1984) Cell 37:767), myc (e.g., Myc1-9E10, Evan et al. (1985) Mol. Cell. Biol. 5:3610-3616), VSV-G, FLAG, and 6-histidine (see, e.g., German Patent No. DE 19507 166).

Another exemplary specific binding pairs includes a cell surface protein and a ligand (e.g., a peptide or polypeptide such as an antibody) that binds to it. The cell surface protein can be specific to a particular cell type or to a cell having a particular property, behavior or disorder. For example, the cell can be a cancer cell, and the antibody can bind specifically to hypoglycosylated MUC1, melanoma differentiation antigen gp100, or CEA1.

Interfaces

Information stored in the database 60 can be accessed in a number of ways. Referring again to FIG. 4, the database 60 can be access through an interface on a user system 240 that communicates across the network 245. For example, the user systems 240 can use a web browser that communicates in XML or HTML with the server 205 to query the database 60.

In one example, the interface includes a top level menu that lets the user decide between a number of available options. The user can choose to query display library members, customize a report, audit the system 200 or projects, perform sequence analysis or other bioinformatics tools, and so on. The user select directs the interface to display a child menu that is dedicated to each particular selection.

One child menu enables the user to choose between possible queries. One type of query activates the hit-picking interface described above and in FIG. 8. Other types of queries enable the user to search by any field of the database 60. For example, a search can be run to identify particular projects (e.g., projects initiated before a particular date), particular clients, particular selection conditions (e.g., selections that used a magnetic particles from a particular manufacturer) and so forth.

Another child menu enables a user to customize styles for electronic reports. The style can be associated with a particular client, operator, or project. The style determines parameters for report formatting, e.g., number of hits per page, use of color, use of graphics, and so forth. The style can also specify the file format of the report (e.g., Microsoft® Office Application such as Microsoft® Excel, Word, or PowerPoint, Postscript, Adobe® Portable Document Format (PDF), HTML, XML, meta-tagged text, text, tab-delimited text, Visual Basic-compatible, and so forth). The file can also be encrypted, or protected (e.g., independently read or write protected). The server 205 can access the style specification in order to format automated reports (see below). The style menu can also be accessed after a search is run from the search menu in order to customize a report of the search results.

The style menu can also be used to customize the display of nucleic acid and/or amino acid sequences. Style specifications for sequences can also be associated with particular libraries. Custom parameters can include coloring particular positions in certain colors, or particular residue types in certain colors. For an amino acid sequence, for example, hydrophobic residues might be indicated in red and hydrophilic residues in blue. In an example which is the display of an antibody sequence, positions corresponding to the framework might be in blue and positions corresponding to complementarity determining regions (CDRs) might be in red. In still another example, the style specifies the display of only particular positions, e.g., only variable positions, or only CDR positions.

Yet another child menu enables the user to audit the system or a project. When this option is triggered, the server 205 queries the user as to the type and extent of audit required. For example, an audit of the system can include a textual display or report that concisely lists active apparati and active projects. Of course, other levels of detail are available. In another example, the system audit is rendered as a graphical view in which apparati are represented as icons and colored according to operational throughput.

A project audit can also be rendered as a graphic with icons positioned on a timeline with reference to the current date, future target dates, and past milestones. The same audit can also be presented in tabular form as text.

Still another child menu enables the user to interface with bioinformatics tools such as those described below.

One type of interface shows a list of some or all display library picks, e.g., from one or more projects, or one or more hit lists. The interface can show one or more fields described herein in any style, e.g., a user-specified style. For example, the interface can show identifiers, amino acids at selected positions, and assay information, e.g., functional assay information such as for a binding or enzymatic assay. Selected sequence features can be identified, e.g., by parsing input sequence information as described below.

The interface can also show a parameter associated with sequence analysis of each displayed library member, e.g., similarity to a reference sequence (e.g., percentage identity or a score), similarity to a consensus sequence, hydrophobicity (e.g., overall or at selected sites), hydrophilicity, pI, charge, molecular weight, predicted Stokes radius, drugability, and so forth. Such parameters and scores can be determined, e.g., an formula, e.g., a empirical, arbitrary, or theoretical formula.

Another parameter indicated on the interface can be a function of two or more fields. For example, one of the parameters can be a specificity ratio, e.g., binding activity to a target divided by binding activity to a non-target (e.g., a molecule that is homologous, but non-identical to a target molecule).

Bioinformatics and Sequence Analysis

A variety of bioinformatics tools can be applied manually or automatically to analyze sequences identified by the display library screening process 10. Examples of such tools include sequence parsing, sequence searching, multiple sequence alignments, and structure modeling.

Sequence Parsing. Data for nucleic acid sequences determined from a nucleic acid sequencing instrument can be parsed. The parsing can be implemented at any time and by any processor, e.g., a processor associated with the sequence instrument, a networked computer system, and so forth. In one embodiment, parsing includes evaluating quality scores of raw sequence data and identifying patterns of nucleotides, e.g., nucleotides at invariant positions in the display vector, or in the displayed protein. In some cases, the nucleotide patterns are sets of codons that encode a particular polypeptide motif.

In one embodiment, the parsing rules are designed to identify relevant sequence features in a library that includes a pool of natural diversity (e.g., natural immunoglobulin variable domain diversity). Such rules can be identified by comparing known members in the protein family, and including library construction considerations, e.g., the use of particular degenerate primers or invariant primers, and so forth. Rules that search natural diversity are typically broad so that all codons encoding a particular conserved amino acid or a particular set of amino acids are identified at the relevant position.

In another embodiment, the parsing rules are designed to identify relevant sequence features in a synthetic library. The synthetic library may include controlled degrees of variation at particular nucleotide positions (e.g., as described herein). The parsing rules can be defined “narrowly” to identify features consistent with the library design. Some libraries which include both natural and synthetic diversity can include both types of rules for each relevant position.

By identifying particular features in a nucleic acid sequence, regions that are varied or that are predicted to participate in a physical interaction (e.g., CDR positions) can be automatically located and highlighted for a user. In addition, nucleic acid sequence encoding invariant regions can be trimmed from the data. For example, vector sequences upstream of the coding region are discarded.

In one implementation, trimmed nucleic acid sequences are compared to each other so that duplicates can rapidly be identified. Library members can be sorted into groups based on their sequence identity. For example, a first group might include all library members (e.g., from an immunoglobulin library) that have a particular light chain sequence are clustered into a group. Members of the group may all be identical or they may include variations in the heavy chain sequence. An interface can indicate the number of groups and the number of members in each group. Other criteria, e.g., other than sequence identity can be used, e.g., groups can be formed based on homology, hydrophobicity, and so forth. The user can view results of a screen as groups and can select a group in order to visualize individual members of the group.

In one example, nucleic acid sequence data for display library members that encode an immunoglobulin variable domain are parsed to identify sequence features that locate the signal sequence, FR1, FR2, FR3, FR4, and the constant region. In many cases the location of these features is non-trivial because CDRs can have varied lengths. One example of features that can be identified in naturally diverse immunoglobulin variable domains are the features in Tables 3 and 4.

TABLE 3
Parsing Immunoglobulin Light Chain Variable Domain

| Rule | Name of Feature |
|---|---|
| FYSH[S\|R]. | Signal |
| QDI[Q\|V].{19}. | FR1_1 |
| QS.{19}. | FR1_2 |
| W.{1,2}Q.{9,10}I. | FR2 |
| G[V\|M\|I].{27,29}Y[Y\|H\|F]C | FR3 |
| FG.G[T\|A].{5} | FR4 |
| [G\|S\|R]QP.{3,4}P\|R.{4,5}P | Tail |

TABLE 4
Parsing Immunoglobulin Heavy Chain Variable Domain

| Rule | Name of Feature |
|---|---|
| .{3}QPA[M\|S]A | Leader Sequence |
| EVQ.+LRLSCAASGFTF[S\|Y] | FR1 |
| .Y.M. | CDR1 |
| WVRQAPGKGLEWVS | FR2 |
| .1.{2}SGG.T.YADSVKG | CDR2 |
| R.{22}EDTA | FR3 |
| .[Y\|C]YCA[R\|K\|S] | FR3 |
| .+WG[R\|K\|Q]G[T\|A] | CDR3 & FR4 |
| .VTVS. | FR4 |
| ASTKGPSVFP | Tail |

The rules in Tables 3 and 4 identifying these features are written using the PERL conventions for string comparison. Symbol=. (dot) Meaning=Any non-space character (used to denote any Amino Acid, or version of stop codon represented by (., q, s, *). Symbol=+Meaning=Any length when used in combination (for instance .+means infinite length of non-space characters in a row). Symbol=[x|y] Meaning=This particular position in the amino acid pattern can be either x or y. Can be used in combination like [xu|yu] meaning two aa following each other named either xu or yu. Anything in between the [ ] symbols is consider a single entity in PERL even though it may be matching multiple amino acids in a row. Example: [xu|yu]{4} means the pattern xu or yu occurring four times in a row like “xuyuxuyu” or some other combination. Symbol={x} Meaning=Preceding pattern is matched exactly x number of times where x is an integer. Example: x{5} will require five x's in a row to match pattern. Symbol={x,y} Meaning=Whatever pattern comes before will match a minimum of x and a maximum of y where x and y are integers. Example: x{1,2} means at least one x and at most two x's in a row to match pattern. Combinations such as x{1,} and x{,4} mean at least one x and at most 4 x's in a row respectively.

In some embodiments, sequences that are not successfully parsed are flagged for either manual review or automatic resequencing. See, e.g., “Automated Information Management”, below.

Sequence Searching. The interface menu can provide an option for performing a nucleic acid or amino acid sequence search using one or more of the sequenced candidate library members. Standard sequence comparison routines such as BLAST (Altschul, et al. (1990) J. Mol. Biol. 215:403-10), FASTA (Pearson (1990) Methods Enzymol 183:63-98)), and CLUSTALW (Thompson et al. (1994) Nucl Acids Res 22:4673-4680) can be used for the comparisons. For example, the comparisons can be executed by modules provided by the GCG® WISCONSIN PACKAGE™ program (Accelrys, San Diego Calif.). An interface can be used to execute the modules so that the analysis can be effected by merely selecting the sequence. The sequence search routines can search one or more of the following databases:

Non Redundant Nucleic acid Sequences (e.g., from GenBank, available from the National Center for Biotechnology Information, National Institutes of Health, Bethesda Md.)

Non-Redundant polypeptide sequences (e.g., from GenBank)

Patented Sequences

Proprietary sequences, such as a collection of other sequenced display library members (e.g., any display library member whose information is stored at the server for all available projects, a given project or a set of projects).

Searching of naturally-occurring and other available sequences may identify common features of biological relevance for activity. Searching of proprietary sequences can identify false positives, e.g., sequences have a propensity for being identified in selection campaigns against unrelated targets.

Multiple Sequence Alignments. Searching can also be used to identify motifs within a collection of hits, e.g., hits for a given project. Pairwise alignments between all hits isolated from a given selection campaign or a given project are executed recursively to produce one or more sequence alignments. For example, the GCG® “pileup” module can be used to attempt to align all such sequences. Phylogenetic techniques, such as the phylogenetic bootstrapping techniques of PHYLIP, can also be used to attempt to force such an alignment (see, e.g., Felsenstein (1989) Cladistics 5:164-166 and on-line resources provided by the University of Washington, Seattle Wash.).

This analysis may identify a motif that is common among ligands, particularly ligands that have at least a threshold activity. The identification of such a motif can be used to design a smaller display library dedicated to densely sampling the sequence space surround the motif (see below).

Searching of external and internal sequence databases can also be automated, e.g., as an automated check described below.

Structure Modeling. This tool can be used to model the three-dimensional coordinates of a display library member. The tool first constructs a model using one of many possible modeling techniques. Then, the tool renders the model as a two- or three-dimensional image on an interface for viewing by a user.

Modeling techniques can rely on standard strategies, such as homology modeling and energy minimization. Methods of computer-aided, homology-based structural prediction are well known and can be automated and performed locally using a desk-top PC or remotely, e.g., by accessing a server hosting the application. One exemplary homology modeling suite is the SWISS-MODEL structural prediction platform (see, e.g., Guex et al. (1999) TiBS 24:364-367; and on-line resources available from EXPASY at Swiss Institute of Bioinformatics, Geneva, Switzerland). Other more sophisticated algorithms, which involve less automation, can also be used. Some prediction platforms, such as Ludi (Biosym Technologies Inc., San Diego, Calif.) and Aladdin (Daylight Chemical Information Systems, Irvine Calif.), are commercially available.

The model can also be docked with the target ligand or substrate. See, e.g., Ewing and Kuntz (1997) J. Comput. Chem. 18:1175-1189.

Automated Information Management

The server 205 can also implement automated checks, e.g., periodically, (e.g., nightly, weekly, etc.) to monitor the many projects and apparati handled by the system 200.

One set of automated checks determines the efficiency of apparati usage for a given interval. For example, the server can determine from event logs whether each apparatus is performing optimally. Increased downtime can generate an automated alert, e.g., by email, to an operator or service technician for the apparatus in question.

Another set of automated checks determines the performance of each library. This analysis can be performed on the level of composite libraries, sublibraries, and individual libraries. The system can determine the number of candidates being identified from each of the libraries and can gather statistics on the success rates of those candidates. Libraries that perform poorly relative to other similarly designed libraries are noted. Operators of the library in question can receive an automated alert indicating possible sub-optimal performance.

Likewise, the server 205 can compare the sequence of candidates obtained from the same library for different projects. If the server identifies a sequence or motif that is overrepresented in candidates isolated against unrelated target compounds, these sequences can be flagged in all projects in which they appear. The flag alerts an operator that the sequence might be a false positive and should be checked for activity towards the unrelated target compounds.

For composite libraries, the server can determine if each sublibrary is performing to expectations. For example, statistics about the number of candidates obtained from each sublibrary are updated and compared against design parameters. Library designers can check the statistics to modify the proportion of sublibraries in preparations of new composite libraries and to control the quality of new library construction.

A third set of automated checks can monitor the progress of projects. The server 205 can compare progress made to date against forecasted milestones entered earlier. The server 205 automatically alerts operators and managers of delays. The server 205 can also modify the forecast based on past progress and information about apparati efficiencies. For example, if downtime is detected, e.g., due to required maintenance or reagent shortages, the forecast is altered and the operators and managers are notified.

A fourth set of automated checks can initiate sequence comparisons and/or multiple sequence alignments of sequenced display library members, e.g., within a project, a screen, or in the entire database. The server 205 can execute these tasks and automatically deliver reports of results to operators. In addition or in an alternative, the server 205 can analyze the results and note trends and deviations from expectations. For example, if the sequences of all sequenced library members can be fitted to a single multiple sequence alignment, this might indicate to a user that a bias was introduced in the screening process or that a common molecular interface is operating.

A fifth set of automated checks can verify the quality of data received by the server 205. For example, each sequence read that is received can be verified using quality parameters, e.g., parameters from PHRED. In another example, data scanned from an assay plate is evaluated, e.g., values for background and control samples can be compared to tolerated ranges.

When the checking routine identifies data that is sub-standard or that meets some criterion, the system can automatically instruct a sample handling device to obtain more data. For example, if a sequence is of poor quality, the system can initiate a request or instructions for re-sequencing. Further, the checking routine can indicate a primer and strand for the sequencing reaction, for example, if the quality deteriorates in a particular region. Likewise, the system can initiate a request or instructions to run an assay again.

When data quality deviates the system can also interface with an instrument or a user to troubleshoot laboratory conditions and reagents. Information collected can be stored in a database that associates information about data quality and experimental conditions. Then, system can be trained (e.g., using neural nets, fuzzy logic, or statistical correlation) to identify or suggest problems when poor quality data is received. For example, if particular sequence reads are correlated with low activity DNA sequencing enzymes (i.e., polymerase), the system can alert a user or instrument to check or provide a new batch of enzyme. Thus, reagents, instruments, samples, and environmental conditions can be automatically monitored by the system.

Bar Coding & Event Auditing

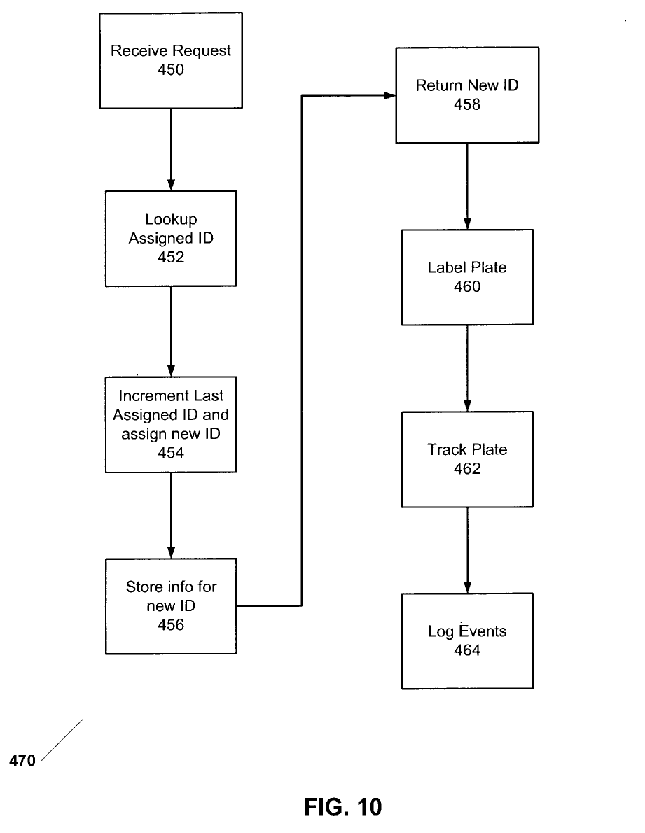

Referring to FIG 10, each multi-well plate is assigned a unique plate identifier, typically, when it is first prepared. This assignment includes requesting 450 a unique identifier from the server 205. The server 205 looks up 452 a table of assigned plate identifiers and, for example, determines the next identifier to be assigned, e.g., by incrementing 454 the highest value identifier. The project name or a project number can be concatenated to the left of the identifier for ease of reference. The server stores 456 information associated with the request in the table of assigned plate identifiers and returns 458 the unique identifier to the plate picking apparatus. The identifier can then be labeled 460 on the multi-well plate using a bar code. The multi-well plate is tracked 462 for each event that it is subjected to. The tracking can include scanning the bar code label prior to and after each event. Instances of the events are communicated to the server and logged 464.

The log can be a table of events. Each event includes an association between the plate identifier for which the event was tracked, an indication of the nature of the event (e.g., “prepared at station 1,” “inoculated at station 2,” and so forth), and information about the time and location of the event. The descriptive information can be coded, e.g., using codes that are identified in a table of event codes.

The overall process 440 enables multi-well plates to be easily and accurately labeled. Further, as all events associated with the screening process 10 are tracked, it is possible to determine the status of a project or the system 200 as a whole. Further, if a multi-well plate is located within the system 200, information about its contents and history are easily retrieved by querying the server 205.

Other types of object identifiers can be used, e.g., instead of bar codes. For example, each plate can include any type of optical, magnetic, electronic, chemical, or physical identifier such as a radio-frequency (RF) tag, a hologram, or an electronic chip.

External Clients

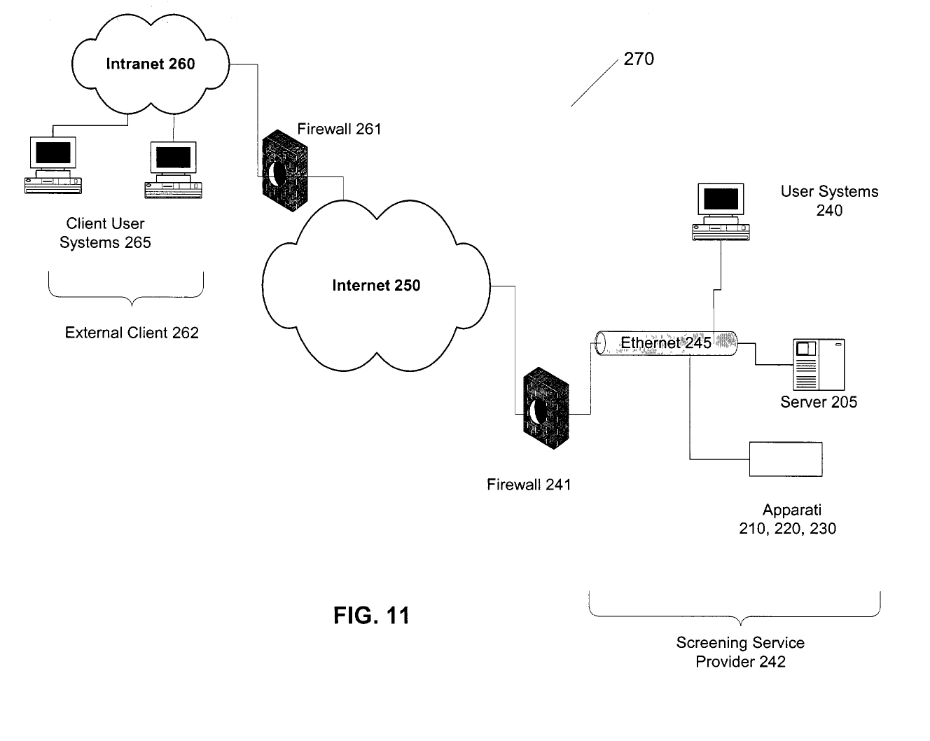

Referring to FIG. 11, an external client 262 commissions a project from a library screening service provider 242. The database 60 is configured to enable individuals associated with the external client 262 to access information. Access can be restricted to 1) authorized individuals at the external client, 2) information associated with the commissioned project, but not projects commissioned by others, 3) information that has been released by an internal user, e.g., for verification and quality control.

For example, an individual at a client user system 265 at the external client 262 can be connected to an intranet 260 that is interfaced with the Internet 250 by a firewall 261. The Internet 250 is used to route communications with the server 205, which is connected to an internal Ethernet 245 at the screening service provider 242. The Ethernet 245 is, likewise, protected by a firewall 241.

The client user system 265 can use standard hypertext transfer protocols to securely communicate with the server 205 across this network. Electronic certificates and passwords are exchanged to authenticate the individual. The authenticated individual can view a menu in a web browser. The menu, for example, enables the individual to view a project summary, request and/or view reports of events, screening hits, and assays results; and communicate with contacts at user systems 240 within the screening service provider 242. Some information, such as reports, can be delivered by e-mail or directly to the web browser. Reports can be formatted for a Microsoft® Office Application such as Microsoft® Excel, Word, or PowerPoint, or for Postscript or Adobe® Portable Document Format (PDF).

The project summary can include a graphical timeline that display target dates for milestones associated with the project and an indication of actual progress. E.g., the timeline can include milestones such as “Screen Library”; “Assay 10,000 Hits”; “Sequencing Best 2,000”; “Screen Library Round 2”; “Recombinant Production”; “Product Verification”; and “Delivery.”

In one implementation, the interface also enables a user at a client user system 265 to communicate with the screening service provider 242, e.g., a manager or administrator at the screening service provider 242. For example, the interface can include a region for the entry of text comments or request. Another interface can allow for selection of graphical or textual indicators of customer satisfaction, or even the entry of data, required parameters, or assay conditions from the client user system. Some or all of this information can be processed automatically, e.g., to configure an assay of display library member hits according to user-entered parameters.

In some implementations, the server 205 can include software configured to manage billing and other accounting information. For example, the database record for the external client 262 in the client table can include fields for billing codes, billing rates and plans, and accounting personnel contacts. When a project is initiated, the billing arrangements are entered into the client entry. During the project, the software can automatically detect when specified milestones are reached and generate an invoice for billing the external client 262. The server 205 can also be interfaced with a business-to-business exchange, such as that commercially available from SAP AG (Walldorf, Germany) for automated transactions.

The software can also track consumables, equipment time, and operator time for each project. This information can be used to bill the external client 262, or for cost-control and cost-efficiency management.

The server can also track deliveries and orders related to operation of the system. In particular when lead candidates are identified, these can be delivered by a courier to the external client 262. Tracking information for the delivery is generated by the server on-line, e.g., with the courier operator. Further orders made by the library screening party 242, e.g., for enzymes, multi-well plates, and other consumables can also be tracked by the server 205 to insure on-time delivery materials needed for each project.

Implementation of Software and Database

The computer-based aspects of the system 200 can be implemented in digital electronic circuitry, or in computer hardware, firmware, software, or in combinations thereof. An apparatus of the invention, e.g., the server 205, can be implemented in a computer program product tangibly embodied in a machine-readable storage device for execution by a programmable processor; and method actions can be performed by a programmable processor executing a program of instructions to perform functions of the invention by operating on input data and generating output. The invention can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object oriented programming language, or in assembly or machine language if desired; and in any case, the language can be a compiled or interpreted language. Suitable processors include, by way of example, both general and special purpose microprocessors. Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks such as, internal hard disks and removable disks; magneto-optical disks; and CD_ROM disks.

Of course, the server 205, for example, can also be distributed among more than one computer.

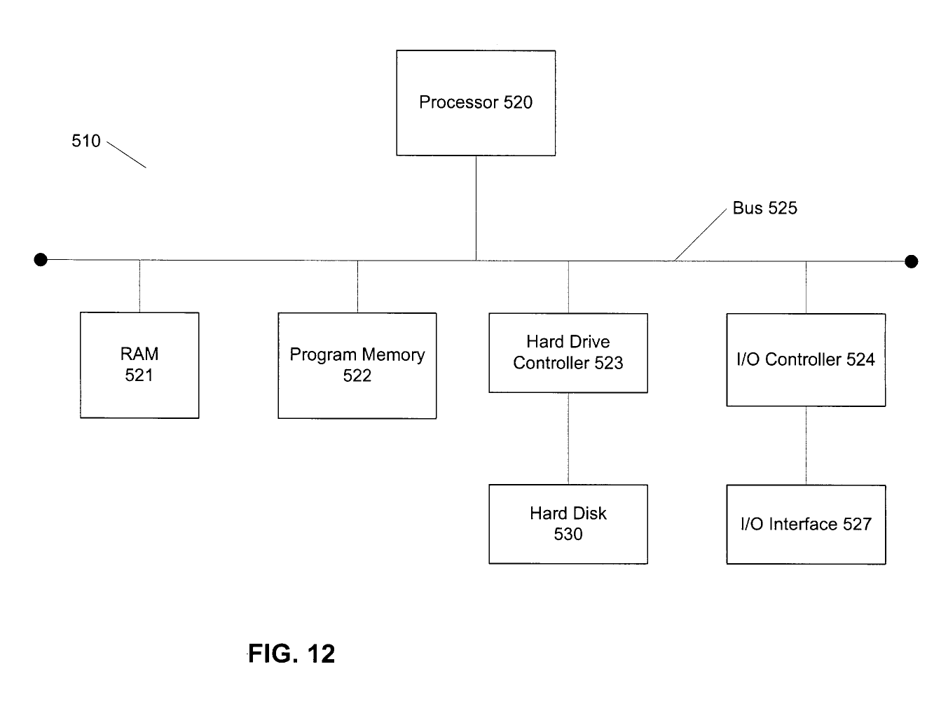

An example of one such type of computer is shown in FIG. 12, which shows a block diagram of a programmable processing system 510 suitable for implementing or performing the apparatus or methods of the invention. The system 510 includes a processor 520, a random access memory (RAM) 521, a program memory 522 (for example, a writable read-only memory (ROM) such as a flash ROM), a hard drive controller 523, and an input/output (I/O) controller 524 coupled by a processor (CPU) bus 525. The system 510 can be preprogrammed, in ROM, for example, or it can be programmed (and reprogrammed) by loading a program from another source (for example, from a floppy disk, a CD-ROM, or another computer).

The hard drive controller 523 is coupled to a hard disk 530 suitable for storing executable computer programs, including programs embodying the present invention, and data including storage. The I/O controller 524 is coupled by means of an I/O bus 526 to an I/O interface 527. The I/O interface 527 receives and transmits data in analog or digital form over communication links such as a serial link, local area network, wireless link, and parallel link.

One non-limiting example of an execution environment includes computers running Windows NT 4.0 (Microsoft) or better or Solaris 2.6 or better (Sun Microsystems) operating systems. Browsers can be Microsoft Internet Explorer version 4.0 or greater or Netscape Navigator or Communicator version 4.0 or greater. Computers for databases and administration servers can include Windows NT 4.0 with a 400 MHz Pentium II (Intel) processor or equivalent using 256 MB memory and 9 GB SCSI drive. Alternatively, a Solaris 2.6 Ultra 10 (400 Mhz) with 256 MB memory and 9 GB SCSI drive can be used.

Post-Processing

Referring again to FIG. 5, the process 300 can include a variety of so-called “post-processing” methods. For example, after the first functional assay 324, the method can include additional assays. These additional assays can differ from the initial set of assays or can be repetitions of them. They can be performed prior to hit-picking 330 or after hit-picking and/or after sequencing 340. Additional assays can be used to obtain information about:

Specificity, e.g., binding to non-target molecules or catalytic activity for non target molecules;

Affinity: apparent Kd's, or kinetic parameters for catalysis;

Binding site or “epitope” (e.g., Competing compounds that differ from the target compound by one or a few epitopes can be used to identify the epitope bound by a display library member)

Stability (e.g., display library members can be pre-treated or assayed under a variety of conditions that probe the stability of polypeptides. Such treatments include, e.g., exposure to chaotropes, pH extremes, and heat.

Biological Activity (e.g., ability to modulate a cellular process such as proliferation, differentiation, apoptosis, cell migration, cell adherence, and so forth).

Physiological Properties (e.g., renal clearance, toxicity, target tissue specificity, and so forth)

Some examples of high throughput functional assays include ELISAs, homogenous assays, and binding to protein arrays.

ELISA (Enzyme-Linked ImmunoSorbent Assay). The binding interaction of a library member for a target can be analyzed using an ELISA assay. For example, the library member is contacted to a microtitre plate whose bottom surface has been coated with the target, e.g., a limiting amount of the target. The plate is washed with buffer to remove substances non-specifically bound to the target and the plate. Then the amount of the library member bound to the plate is determined by probing the plate with an antibody that recognizes library members. For example, in the case of a display library member, the antibody can recognize a region that is constant among all display library members, e.g., for a phage display library member, a major phage coat protein. The antibody is linked to an enzyme such as alkaline phosphatase, which produces a colorimetric product when appropriate substrates are provided. In some cases, the amount of colorimetric product produced can be determined by an optical reader that measures the optical density at the wavelength absorbed by the colorimetric product.

Some post-processing analyses can include variations of the ELISA method that glean the additional information listed above (e.g., specificity, etc.). For these analyses, ELISAs can include varying the amount of input display library member, the amount of target compound, the amount of a competitor, the pH, the ionic strength, the temperature, the presence of a reducing agent, or the presence of a protease.

ELISAs can also be performed in a “kinetic mode.” In this mode, immediately after set up, an ELISA assay is transferred to a liquid handling station which removes unbound display library members from solution at set time periods. In another implementation, a competing amount of the target compound is added for set time periods. The competing target compound is prevented from binding the assay plate and is present in saturating amounts so that dissociating display library members do not reassociate to the target compound that is bound to the plate. Results from this binding assay provide information about the off rate for binding.

Homogeneous Assays. The binding interaction with a target can also be analyzed using a homogenous assay, i.e., after all components of the assay are added, additional fluid manipulations are not required. Typically, a display library member is modified to include one label that is required for the assay and the target compound is modified to include the other label. The label can be covalently or non-covalently attached. For example, an antibody bearing the label can be used to attach the label to a phage display library ember.

Fluorescence resonance energy transfer (FRET) can be used as a homogenous assay (see, for example, Lakowicz et al., U.S. Pat. No. 5,631,169; Stavrianopoulos, et al., U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FRET binding event can be conveniently measured through standard fluorometric detection means well known in the art (e.g., using a fluorimeter). By titrating the amount of the first or second binding molecule, a binding curve can be generated to estimate the equilibrium binding constant. Another homogenous assay uses the AlphaScreen™ technology available from Biosignal Packard (Montreal, Quebec). Donor beads that generate singlet oxygen when excited by a laser are attached to one member of the binding assay, e.g., the display library member. Acceptor beads which emit light when contacted by singlet oxygen that diffuses from the donor bead are attached to the other member of the binding assay, e.g., to the target compound. This system and FRET are examples of proximity assays.

Protein Arrays. Proteins from each isolated display library member can be immobilized on a solid support, for example, on a bead or an array. For a protein array, each of the polypeptides is immobilized at a unique address on a support. Typically, the address is a two-dimensional address.

In some implementations, the display library member itself is amplified and disposed on the array. For example, cells or phage can be grown directly on a filter that is used as the array. In other implementations, recombinant protein production is used to produce at least partially purified samples of the protein. The partially purified (or pure) samples are disposed on the array.

Methods of producing protein arrays are described, e.g., in De Wildt et al. (2000) Nat. Biotechnol. 18:989-994; Lueking et al. (1999) Anal. Biochem. 270:103-111; Ge (2000) Nucleic Acids Res. 28, e3, I-VII; MacBeath and Schreiber (2000) Science 289:1760-1763; WO 01/40803 and WO 99/51773A1. Polypeptides for the array can be spotted at high speed, e.g., using commercially available robotic apparati, e.g., from Genetic MicroSystems or BioRobotics. The array substrate can be, for example, nitrocellulose, plastic, glass, e.g., surface-modified glass. For example, the array can be an array of antibodies, e.g., as described in De Wildt, supra.

A protein array can be contacted with a labeled target to determine the extent of binding of the target to each immobilized protein from the diversity strand library. Information about the extent of binding at each address of the array can be stored as a profile, e.g., in a computer database. The protein array can be produced in replicates and used to compare binding profiles, e.g., of a target and a non-target. Thus, protein arrays can be used to identify individual members of the diversity strand library that have desired binding properties with respect to one or more molecules.

Recombinant Production. As mentioned above, some post-processing analyses require partially purified or purified samples of the displayed polypeptide. For these analyses, recombinant polypeptide production techniques are used to prepare the samples.

For example, the server 205 can include an interface that enables an operator to select candidate display library members for recombinant production. As described above, the interface can include check-boxes, pull-down menus, or search queries that can be used to select candidate display library members. Based on user selections, the server 205 can direct a sample handling device to prepare the selected display library members for recombinant polypeptide production.

The sample handling device can process the selected library members in an automated cloning process. For example, the device can perform manipulations (e.g., PCR, other amplification, plasmid, or single-stranded nucleic acid preparation) to obtain nucleic acid that encodes the relevant displayed polypeptide of each library member, and insert the nucleic acids into a new vector or a new context for a downstream-application, such as recombinant production. Of course, automated cloning can be used to reformat library members for other purposes, e.g., sequencing, archiving, transgenic animal production, gene deletion, and so forth.

In cases where the displayed polypeptide is displayed as a fusion to a phage member coat protein or fragment thereof and a suppressible stop codon is included in the nucleic acid encoding the fusion, the sample handling device can transfer nucleic acid encoding the displayed polypeptide into a non-suppressing bacterial strain. This implementation does not require recloning or other reformatting of library nucleic acids.

In another example, the sample handling device can assemble reactions for the amplification of nucleic acid encoding the variant region of each selected display polypeptide. The reactions can be transferred to an amplification conditions, e.g., in a thermal cycler. Then, amplified fragments can be isolated and cloned into an expression vector, e.g., a eukaryotic (e.g., mammalian, plant, or fungal) or prokaryotic expression vector.

In yet another example, the nucleic acid encoding the variant region (or the entire displayed polypeptide) is amplified with primers that include terminal recombination sites. Such sites can also be designed in the display vector, in which case no amplification is needed. The nucleic acid is inserted into an expression vector using recombination, e.g., in vivo recombination or in vitro recombination (e.g., recombinational cloning).

Methods for recombinational cloning are described, e.g., in U.S. Pat. No. 5,888,732; Walhout et al. (2000) Science 287:116; and Liu et al. (1998) Curr. Biol. 8(24):1300-9. Recombinational cloning exploits the activity of certain enzymes that cleave DNA at specific sequences and then rejoin the ends with other matching sequences during a single concerted reaction. The recombination reaction can take place in vitro. After which, the reaction mixture is transformed into an appropriate bacterial host strain. The target vector can contain a gene that is toxic to bacteria that is located between the recombination sites such that excision of the toxic gene is required during recombination. Thus, the cloning products that are viable in bacteria under the appropriate selection are almost exclusively the desired construct. In practice, the efficiency of cloning the desired product approaches 95 to 100%. This high efficiency enables the process to be performed automatically, e.g., by robots with minimal supervision.

After automated cloning (e.g., sub-cloning), the cloned selected library members can be verified in a high-throughput format or screened, e.g., without verification.

A number of types of cells may act as suitable host cells for expression of the proteins encoded by the selected library members. Scopes (1994) Protein Purification: Principles and Practice, New York:Springer-Verlag provides a number of general methods for purifying recombinant (and non-recombinant) proteins. The method include, e.g., ion-exchange chromatography, size-exclusion chromatography, affinity chromatography, selective precipitation, dialysis, and hydrophobic interaction chromatography. These methods can be adapted for devising a purification strategy for the proteins of the selected library members, e.g., in parallel. In particular, purification handles such as the hexa-histidine tag and epitope tags can be used. For antibodies and antibody fragments, antibody binding proteins such as protein A, L, or G can be used.

Synthetic Production. In the case of polypeptides of less than 70 amino acids, and more typically of less than 30 amino acids, the polypeptides identified by the display library screen can be synthesized, e.g., using t-BOC/FMOC based synthesis. The server 205 can include an interface that enables an operator to select display library members for peptide synthesis. A string representing the amino acid sequence of each selected member is then transmitted (locally or remotely) to an automated peptide synthesizer. The synthesizer then produces the peptide and disposes it in a bar-coded labeled container, e.g., a well of a multi-well plate or a stand-alone container. These containers can also be tracked by the system. Optionally, the synthesis is followed by HPLC purification of the peptide and mass spectroscopy verification, either under manual or automation direction.

Surface Plasmon Resonance (SPR). Displayed polypeptides can be assayed for binding the target using SPR. SPR or real-time Biomolecular Interaction Analysis (BIA) detects biospecific interactions in real time, without labeling any of the interactants (e.g., BIAcore). Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander, S. and Urbaniczky, C. (1991) Anal. Chem. 63:2338-2345; Szabo et al. (1995) Curr. Opin. Struct. Biol. 5:699-705 and on-line resources available from BIACore International AB (Uppsala, Sweden).

Information from SPR can be used to provide an accurate and quantitative measure of the equilibrium dissociation constant (K_{d}), and kinetic parameters, including Kₒₙ and K_{off}, for the binding of a biomolecule to a target. Such data can be used to compare different biomolecules. For example, proteins selected from a display library can be compared to identify individuals that have high affinity for the target or that have a slow K_{off}. This information can also be used to develop structure-activity relationship (SAR) if the biomolecules are related. For example, if the proteins are all mutated variants of a single parental antibody or a set of known parental antibodies, variant amino acids at given positions can be identified that correlate with particular binding parameters, e.g., high affinity and slow K_{off}.

Additional methods for measuring binding affinities include fluorescence polarization (FP) (see, e.g., U.S. Pat. No. 5,800,989), nuclear magnetic resonance (NMR), and binding titrations (e.g., using fluorescence energy transfer).

Biological Assays. Recombinantly produced displayed polypeptides can be assayed for biological activity. In one example, the polypeptides are fused to the Fc effector domain of an immunoglobulin. The displayed polypeptide can itself be a fragment of an immunoglobulin, e.g., a single chain immunoglobulin or a Fab fragment. However, the display polypeptide may not be a fragment of an immunoglobulin.

Display library members fused to Fc effector domains can be assayed for cytotoxicity in two modes: antibody-dependent cell-mediated cytotoxicity (ADCC) or complement dependent cytotoxicity (CDC). These assays are routine in the art.

Numerous cell culture assays for differentiation and proliferation are known in the art. Some examples are as follows:

Assays for embryonic stem cell differentiation (which will identify, among others, proteins that influence embryonic differentiation hematopoiesis) include, e.g., those described in: Johansson et al. (1995) Cellular Biology 15:141-151; Keller et al. (1993) Molecular and Cellular Biology 13:473-486; McClanahan et al. (1993) Blood 81:2903-2915.

Assays for lymphocyte survival/apoptosis (which will identify, among others, proteins that prevent apoptosis after superantigen induction and proteins that regulate lymphocyte homeostasis) include, e.g., those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233 243, 1991; Zacharchuk, Journal of Immunology 145:4037 4045, 1990; Zamai et al., Cytometry 14:891-897, 1993; Gorczyca et al., International Journal of Oncology 1:639-648, 1992.

Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad. Sci. USA 88:7548-7551, 1991.

Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:4062-4069, 1993; Huang et al., Science 264:961-965, 1994; Macatonia et al., Journal of Experimental Medicine 169:1255-1264, 1989; Bhardwaj et al., Journal of Clinical Investigation 94:797-807, 1994; and Inaba et al., Journal of Experimental Medicine 172:631-640, 1990.

Assays for T-cell or thymocyte proliferation include without limitation those described in: Current Protocols in Immunology, Ed by J. E. Coligan, A. M. Kruisbeek, D. H. Margulies, E. M. Shevach, W. Strober, Pub. Greene Publishing Associates and Wiley Interscience (Chapter 3, —Tn vitro assays for Mouse Lymphocyte Function 3.1-3.19; Chapter 7, Immunologic studies in Humans); Takai et al., J. Immunol. 137:3494 3500, 1986; Bertagnolli et al., J. Immunol. 145:1706 1712, 1990; Bertagnolli et al., Cellular Immunology 133:327-341, 1991; Bertagnolli, et al., I. Immunol. 149:3778-3783, 1992; Bowman et al., I. Immunol. 152:1756-1761, 1994.

Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. Coligan eds. Vol 1 pp. 3.12.1-3.12.14, John Wiley and Sons, Toronto. 1994; and Measurement of mouse and human interleukin gamma., Schreiber, R. D. In Current Protocols in Immunology., Coligan eds. Vol 1 pp. 6.8.1-6.8.8, John Wiley and Sons, Toronto. 1994.

Assays for proliferation and differentiation of hematopoietic and lymphopoietic cells include, without limitation, those described in: Measurement of Human and Murine Interleukin 2 and Interleukin 4, Bottomly, K., Davis, L. S. and Lipsky, P. E. In Current Protocols in Immunology. J. E. e.a. Coligan eds. Vol 1 pp. 6.3.1-6.3.12, John Wiley and Sons, Toronto. 1991; deVries et al., J. Exp. Med. 173:1205 1211, 1991; Moreau et al., Nature 336:690-692, 1988; Greenberger et al., Proc. Natl. Acad. Sci. U.S.A. 80:2931-2938, 1983; Measurement of mouse and human interleukin-6, Nordan, R. In Current Protocols in Immunology. J. E. e.a. Coligan eds. Vol 1 pp. 6.6.1 6.6.5, John Wiley and Sons, Toronto. 1991; Smith et al., Proc. Natl. Aced. Sci. U.S.A. 83:1857-1861, 1986; Measurement of human Interleukin-11, Bennett, F., Giannotti, J., Clark, S. C. and Turner, K. J. In Current Protocols in Immunology. Coligan eds. Vol 1 pp. 6.15.1 John Wiley and Sons, Toronto. 1991.

Assays for T-cell clone responses to antigens (which will identify, among others, proteins that affect APC-T cell interactions as well as direct T-cell effects by measuring proliferation and cytokine production) include, without limitation, those described in: Current Protocols in Immunology, Ed by J. E. Coligan, A. M. Kruisbeek, D. H. Margulies, E. M. Shevach, W Strober, Puh. Greene Publishing Associates and Wiley-Interscience (Chapter 3, In vitro assays for Mouse Lymphocyte Function; Chapter 6, Cytokines and their cellular receptors; Chapter 7, Immunologic studies in Humans); Weinberger et al., Proc. Natl. Acad. Sci. USA 77:6091-6095, 1980; Weinberger et al., Eur. J. Immun. 11:405-411, 1981; Takai et al., J. Immunol. 137:3494-3500, 1986; Takai et al., J. Immunol. 140:508-512, 1988.

Other assays, for example, can determine biological activity with respect to endothelial cell behavior, nerve cell growth, nerve cell migration, spermatogenesis, oogenesis, apoptosis, endocrine signaling, glucose metabolism, amino acid metabolism, cholesterol metabolism, erythropoiesis, thrombopocisis, and so forth.

In vivo Assays. Proteins identified by the display library can also be evaluated in in vivo assays, e.g., by administering the phage display member expressing the protein, or the protein itself (e.g., isolated from the phage) to an organism, e.g., an invertebrate (e.g., nematode, Drosophila) or vertebrate, (e.g.,. a mammal such as a mouse, rat, dog, cow, goat, primate, or human). The organism can also be a model for a particular disease, e.g., a nude mouse xenografted with human tumors. One or more parameters of the organism can be monitored. Information about the parameters can be entered into the database. Exemplary parameters include vital signs, resistance to disease, resistance to stress, activity, renal clearance of the introduced protein, circulating levels of the introduced protein, localization of the introduced protein, and so forth.

In a related embodiment, the protein is expressed using a heterologous nucleic acid in an organism. For example, the nucleic acid encoding the heterologous nucleic acid can be introduced as a transgene or as a DNA vaccine.

These methods can be used to collect data about the toxicity, efficacy, and specificity of one or more proteins selected from a library. The data can be stored in records that are associated with (e.g., referenced) to other information about selected library members. The data can be used to derive structure activity relationships for the proteins.

Display Libraries

A display library is a collection of entities; each entity includes an accessible, diverse polypeptide component and a recoverable component that encodes or identifies the polypeptide component. The polypeptide component can be of any length, e.g., from three amino acids to over 300 amino acids. A variety of formats can be used for display.

Phage Display. One format utilizes viruses, particularly bacteriophages. This format is termed “phage display.” The varied polypeptide component is typically covalently linked to a bacteriophage coat protein or domain thereof. The linkage can be produced by a translational fusion encoded by a nucleic acid, and joining the varied polypeptide and the invariant bacteriophage coat protein or domain thereof. The linkage can also include a flexible peptide linker, a protease site, or an amino acid incorporated as a result of suppression of a stop codon. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; WO 94/05781; WO 00/70023; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al. (1996) Methods Enzymol. 267:129-49; Hoogenboom et al. (1991) Nuc Acid Res 19:4133-4137; and Barbas et al. (1991) PNAS 88:7978-7982. It is also possible to display multi-chain proteins, e.g., Fabs (see below). Further, the varied polypeptide component can be attached by a non-covalent interaction (e.g., fos-jun dimerization) or a non-peptide covalent bond (e.g., a disulfide linkage).

Phage display systems have been developed for filamentous phage (phage fl, fd, and M13) as well as other bacteriophage (e.g. T7 bacteriophage and lambdoid phages; see, e.g., Santini (1998) J. Mol. Biol. 282:125-135; Rosenberg et al. (1996) Innovations 6:1-6; Houshmand et al. (1999) Anal Biochem 268:363-370). The filamentous phage display systems typically use fusions to a minor coat protein, such as gene III protein, and gene VIII protein, a major coat protein, but fusions to other coat proteins such as gene VI protein, gene VII protein, gene IX protein, or domains thereof can also been used (see, e.g., WO 00/71694). In a preferred embodiment, the fusion is to a domain of the gene III protein, e.g., the anchor domain or “stump,” (see, e.g., U.S. Pat. No. 5,658,727 for a description of the gene III protein anchor domain).

The valency of the peptide component can also be controlled. Cloning of the sequence encoding the peptide component into the complete phage genome results in multivariant display since all replicates of the gene III protein are fused to the peptide component. For reduced valency, a phagemid system can be utilized. In this system, the nucleic acid encoding the peptide component fused to gene III is provided on a plasmid, typically of length less than 700 nucleotides. The plasmid includes a phage origin of replication so that the plasmid is incorporated into bacteriophage particles when bacterial cells bearing the plasmid are infected with helper phage, e.g., M13K01. The helper phage provides an intact copy of gene III and other phage genes required for phage replication and assembly. The helper phage has a defective origin such that the helper phage genome is not efficiently incorporated into phage particles relative to the plasmid that has a wild type origin.

Bacteriophage displaying the peptide component can be grown and harvested using standard phage preparatory methods, e.g. PEG precipitation from growth media.

After selection of individual display phages, the nucleic acid encoding the selected peptide components, by infecting cells using the selected phages. Individual colonies or plaques can be picked, the nucleic acid isolated and sequenced.

Cell-based Display. In still another format the library is a cell-display library. Proteins are displayed on the surface of a cell, e.g., a eukaryotic or prokaryotic cell. Exemplary prokaryotic cells include E. coli cells, B. subtilis cells, spores (see, e.g., Lu et al. (1995) Biotechnology 13:366). Exemplary eukaryotic cells include yeast (e.g., Saccharomyces cerevisiae, Schizosaccharomyces pombe, Hanseula, or Pichia pastoris). Yeast surface display is described, e.g., in Boder and Wittrup (1997) Nat. Biotechnol. 15:553-557.

In one embodiment, varied nucleic acid sequences are cloned into a vector for yeast display. The cloning joins the varied sequence with a domain (or complete) yeast cell surface protein, e.g., Flo1, a-agglutinin, α-agglutinin, or fragments derived thereof e.g. Aga2p, Aga1p. A domain of these proteins can anchor the polypeptide encoded by the diversified nucleic acid sequence by a GPI-anchor (e.g. a-agglutinin, α-agglutinin, or fragments derived thereof e.g. Aga2p, Aga1p), by a transmembrane domain (e.g., Flo1). The vector can be configured to express two polypeptide chains on the cell surface such that one of the chains is linked to the yeast cell surface protein. For example, the two chains can be immunoglobulin chains.

Peptide-Nucleic Acid Fusions. Another format utilizes peptide-nucleic acid fusions. Polypeptide-nucleic acid fusions can be generated by the in vitro translation of mRNA that include a covalently attached puromycin group, e.g., as described in Roberts and Szostak (1997) Proc. Natl. Acad. Sci. USA 94:12297-12302, and U.S. Pat. No. 6,207,446. The mRNA can then be reverse transcribed into DNA and crosslinked to the polypeptide.

Ribosome Display. RNA and the polypeptide encoded by the RNA can be physically associated by stabilizing ribosomes that are translating the RNA and have the nascent polypeptide still attached. Typically, high divalent Mg²⁺ concentrations and low temperature are used. See, e.g., Mattheakis et al. (1994) Proc. Natl. Acad. Sci. USA 91:9022 and Hanes et al. (2000) Nat Biotechnol. 18:1287-92; Hanes et al. (2000) Methods Enzymol. 328:404-30. and Schaffitzel et al. (1999) J Immunol Methods. 231(1-2):119-35.

Other Display Formats. Yet another display format is a non-biological display in which the polypeptide component is attached to a non-nucleic acid tag that identifies the polypeptide. For example, the tag can be a chemical tag attached to a bead that displays the polypeptide or a radiofrequency tag (see, e.g., U.S. Pat. No. 5,874,214).

Display technology can be used to obtain specific ligands, e.g., antibody ligands, particular epitopes of a target. This can be done, for example, by using competing non-target molecules that lack the particular epitope or are mutated within the epitope, e.g., with alanine. Such non-target molecules can be used in a negative selection procedure as described below, as competing molecules when binding a display library to the target, or as a pre-elution agent, e.g., to capture in a wash solution dissociating display library members that are not specific to the target.

Antibody

In one embodiment, the display library is screened to identify an immunoglobulin or immunoglobulin fragment. An “immunoglobulin domain” refers to a domain from the variable or constant domain of immunoglobulin molecules. An “immunoglobulin superfamily domain” refers to a domain that has a three-dimensional structure related to an immunoglobulin domain, but is from a non-immunoglobulin molecule. Immunoglobulin domains and immunoglobulin superfamily domains typically contains two β-sheets formed of about seven β-strands, and a conserved disulphide bond (see, e.g., A. F. Williams and A. N. Barclay 1988 Ann. Rev Immunol. 6:381-405). Proteins that include domains of the Ig superfamily domains include T cell receptors, CD4, platelet derived growth factor receptor (PDGFR), and intercellular adhesion molecule (ICAM).

An embodiment of immunoglobulin scaffolds is an antibody, particularly an antigen-binding fragment of an antibody. The term “antibody,” as used herein, refers to an immunoglobulin molecule or an antigen-binding portion thereof. A typical antibody includes two heavy (H) chain variable regions (abbreviated herein as VH), and two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). Each VH and VL is composed of three CDR's and four FRs, arranged from amino-terminus to carboxy-terminus in the following order: FR1, CDR1, FR2, CDR2, FR3, CDR3, FR4.

In a display library of immunoglobulin domains, each of these regions can be varied, e.g., with synthetic or natural diversity. The variation can be introduced into an immunoglobulin variable domain, e.g., in the region of one or more of CDR1, CDR2, CDR3, FR1, FR2, FR3, and FR4, referring to such regions of either and both of heavy and light chain variable domains. In one embodiment, variation is introduced into all three CDRs of a given variable domain. In another preferred embodiment, the variation is introduced into CDR1 and CDR2, e.g., of a heavy chain variable domain. Any combination is feasible.

An antibody can also include a constant region as part of a light or heavy chain. Light chains can include a kappa or lambda constant region gene at the COOH-terminus. Heavy chains can include, for example, a gamma constant region (IgG1, IgG2, IgG3, IgG4; encoding about 330 amino acids).

The term “antigen-binding fragment” of an antibody (or simply “antibody portion,” or “fragment”), as used herein, refers to one or more fragments of a full-length antibody that retain the ability to specifically bind to a target. Examples of antigen-binding fragments include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding fragment” of an antibody.

If necessary, the display library screening methods described herein can include automatically (e.g., using robotic-driven nucleic acid manipulations) transfer an antigen binding domain from one format to anther, e.g., from Fab to Ig or from scFv to Fab, and so forth.

Peptide and Scaffold Domain Variation

In one embodiment, a nucleic acid variation method described herein is used to vary a nucleic acid encoding a peptide, e.g., a peptide ligand that specifically binds to a target or, generally, to vary a nucleic acid encoding any proteinaceous domain, e.g., a domain that binds to a target or participates in binding to a target. The peptide ligand or other target-binding ligand be identified using a display library, e.g., as described below.

Synthetic Peptides. The binding ligand can include an artificial peptide of 32 amino acids or less, that independently binds to a target molecule. Some synthetic peptides can include one or more disulfide bonds. Other synthetic peptides, so-called “linear peptides,” are devoid of cysteines. Synthetic peptides may have little or no structure in solution (e.g., unstructured), heterogeneous structures (e.g., alternative conformations or “loosely structured), or a singular native structure (e.g., cooperatively folded). Some synthetic peptides adopt a particular structure when bound to a target molecule. Some exemplary synthetic peptides are so-called “cyclic peptides” that have at least disulfide bond, and, for example, a loop of about 4 to 12 non-cysteine residues. Many exemplary peptides are less than 28, 24, 20, or 18 amino acids in length.

Peptide sequences that independently bind a molecular target can be selected from a display library or an array of peptides. After identification, such peptides can be produced synthetically or by recombinant means. The sequences can be incorporated (e.g., inserted, appended, or attached) into longer sequences.

An exemplary phage display displays a short, variegated exogenous peptide on the surface of M13 phage. The peptide display library can be synthesized from synthetic oligonucleotides that are designed to have between 4 and 30 varied codon positions, e.g., a segment of 4, 5, 6, 7, 8, 10, 11, or 12 varied codons, flanked by codons for cysteine residues (or complement thereof). The pairs of cysteines are believed to form stable disulfide bonds, yielding a cyclic display peptide. The oligonucleotides can be cloned into a format suitable for display, e.g., so that the varied peptides are displayed at the amino terminus of protein III on the surface of the phage. For example, to produce a loop of four amino acids in a 12 amino acid long sequence, a library is constructed using a template sequence that includes three varied codon positions, a codon encoding cysteine, four varied codon positions, a codon encoding cysteine, and three varied codon positions. The varied codon positions can include a codon encoding any amino acid except cysteine. Such variation can be generated using trinucleotide subunits for nucleic acid synthesis. The patterning and extent of variation can also be precisely controlled, e.g., to generate loops of other sizes and compositions. Cysteine can be omitted altogether to prepare linear peptides. For example, the Lin20 library was constructed to display a single linear peptide in a 20-amino acid template. The amino acids at each position in the template were varied to permit any amino acid except cysteine (Cys).

The techniques discussed in Kay et al., Phage Display of Peptides and Proteins: A Laboratory Manual (Academic Press, Inc., San Diego 1996) and U.S. Pat. No. 5,223,409 are useful for preparing a library of potential binders corresponding to the selected parental template. The libraries described above can be prepared according to such techniques, and screened, e.g., as described above, for peptides that bind to a particular molecular target.

After one or more peptides are selected, template nucleic acids encoding the one or more peptides (or complements thereof) can be prepared. These peptides can be varied in a controlled manner by annealing a diverse set of oligonucleotides, e.g., the oligonucleotides used to construct the original library, under conditions such that only a subset of the oligonucleotides bind. The hybridization conditions favor the annealing oligonucleotides that encode a sequence that has some similarity to the template nucleic acid, so that at least some codons are retained from the originally selected peptides. Diversified nucleic acids that incorporate the annealed oligonucleotides are synthesized to prepare a secondary display library of peptides. In some implementations (e.g., for peptides less than 12 amino acids), it may not be necessary to extend these oligonucleotides, but merely to ligate them to a nucleic acid encoding an invariant sequence (e.g., the anchor protein). Thus, in these implementations, copying of the template strand is not required. For example the oligonucleotide mixture may be retrieved by denaturation of the oligonucleotide-template hybrids and directly cloned on the basis of complementary regions bordering the area of diversity, or after PCR of the retained oligonucleotides. Alternatively the mutant strands are rescued via a Kunkel mutagenesis procedure as described earlier.

An advantage of such mutagenesis procedure is that it is not necessary to characterize the sequences of individual clones, but that whole collections of selected populations can be mutagenized, even without understanding the genetic complexity of the selected population. Thus in one application the prior identification of a consensus sequence is not required. This approach will allow the affinity selection of clones that do not follow a particular consensus as defined after the first round of selection/screening/analysis, and are rare in the initially selected population; often frequency and consensus considerations are used to delete such clones for further analysis or maturation. When this strategy of mutagenesis by hybridization is applied for multiple rounds and carried out under increasing stringency (e.g., one or more of: increased stringency hybridization conditions, thereby gradually reducing the number of mutations introduced; and increased stringency selection, e.g. gradually increasing the stringency of washing when selection for binding to antigen), it is expected that the initial peptide or protein sequence is iteratively matured. The focused access of sequence space can be particularly useful.

Other Exemplary Scaffolds. Other exemplary scaffolds that can be variegated to produce a protein that binds to serum albumin and a particular target can include: extracellular domains (e.g., fibronectin Type III repeats, EGF repeats, T-cell receptors, MHC proteins); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (including inactivated proteases), RNase; chaperones, e.g., thioredoxin, and heat shock proteins; and intracellular signaling domains (such as SH2 and SH3 domains) and antibodies (e.g., Fab fragments, single chain Fv molecules (scFV), single domain antibodies, camelid antibodies, and camelized antibodies); T-cell receptors and MHC proteins.

In many embodiments, the scaffold may be less than 50 amino acids in length. Examples of small scaffolding domains include: Kunitz domains (about 58 amino acids, 3 disulfide bonds), Cucurbida maxima trypsin inhibitor domains (about 31 amino acids, 3 disulfide bonds), domains related to guanylin (about 14 amino acids, 2 disulfide bonds), domains related to heat-stable enterotoxin IA from gram negative bacteria (about 18 amino acids, 3 disulfide bonds), EGF domains (about 50 amino acids, 3 disulfide bonds), kringle domains (about 60 amino acids, 3 disulfide bonds), fungal carbohydrate-binding domains (about 35 amino acids, 2 disulfide bonds), endothelin domains (about 18 amino acids, 2 disulfide bonds), zinc finger domain (no disulfide bonds, a chelated zinc atom), and Streptococcal G IgG-binding domain (about 35 amino acids, no disulfide bonds).

U.S. Pat. No. 5,223,409 also describes a number of so-called “mini-proteins,” e.g., mini-proteins modeled after oc-conotoxins (including variants GI, GII, and MI), mu-(GIIIA, GIIIB, GIIIC) or OMEGA-(GVIA, GVIB, GVIC, GVIIA, GVIIB, MVIIA, MVIIB, etc.) conotoxins. U.S. Pat. No. 6,423,498 describes an exemplary library of varied Kunitz domains and methods for constructing such a library.

As described above for peptide and immunoglobulin domains, after a domain is selected for a particular property, a template nucleic acid encoding it (and optionally other such domains) can be prepared and then varied by annealing diverse oligonucleotides, e.g., synthetic oligonucleotides or oligonucleotides derived from a natural source. The hybridization conditions are controlled to favor the annealing oligonucleotides that encode a sequence that has some similarity to the template nucleic acid, so that at least some codons are retained from the originally selected domains. A secondary display library can then be prepared and screened.

Appropriate criteria for evaluating a scaffolding domain can include: (1) amino acid sequence, (2) sequences of several homologous domains, (3) 3-dimensional structure, and/or (4) stability data over a range of pH, temperature, salinity, organic solvent, oxidant concentration. In one embodiment, the scaffolding domain is a small, stable protein domains, e.g., a protein of less than 100, 70, 50, 40 or 30 amino acids. The domain may include one or more disulfide bonds or may chelate a metal, e.g., zinc.

Diversity

Display libraries include variation at one or more positions in the displayed polypeptide. The variation at a given position can be synthetic or natural. For some libraries, both synthetic and natural diversity are included.

Synthetic Diversity. Libraries can include regions of diverse nucleic acid sequence that originate from artificially synthesized sequences. Typically, these are formed from degenerate oligonucleotide populations that include a distribution of nucleotides at each given position. The inclusion of a given sequence is random with respect to the distribution. One example of a degenerate source of synthetic diversity is an oligonucleotide that includes NNN wherein N is any of the four nucleotides in equal proportion.

Synthetic diversity can also be more constrained, e.g., to limit the number of codons in a nucleic acid sequence at a given trinucleotide to a distribution that is smaller than NNN. For example, such a distribution can be constructed using less than four nucleotides at some positions of the codon. In addition, trinucleotide addition technology can be used to further constrain the distribution.

So-called “trinucleotide addition technology” is described, e.g., in Virnekas et al. (1994) Nucl Acids Res 22:5600-7. Oligonucleotides are synthesized on a solid phase support, one codon (i.e., trinucleotide) at a time. The support includes many functional groups for synthesis such that many oligonucleotides are synthesized in parallel. The support is first exposed to a solution containing a mixture of the set of codons for the first position. The unit is protected so additional units are not added. The solution containing the first mixture is washed away and the solid support is deprotected so a second mixture containing a set of codons for a second position can be added to the attached first unit. The process is iterated to sequentially assemble multiple codons. Trinucleotide addition technology enables the synthesis of a nucleic acid that at a given position can encoded a number of amino acids. The frequency of these amino acids can be regulated by the proportion of codons in the mixture. Further the choice of amino acids at the given position is not restricted to quadrants of the codon table as is the case if mixtures of single nucleotides are added during the synthesis.

Natural Diversity. Libraries can include regions of diverse nucleic acid sequence that originate (or are synthesized based on) from different naturally-occurring sequences.

An example of natural diversity that can be included in a display library is the sequence diversity present in immune cells. This diversity includes variation of antibodies, MHC-complexes and T cell receptors. Some examples of immune cells are B cells and T cells. The immune cells can be obtained from, e.g., a human, a primate, mouse, rabbit, camel, or rodent. In one example, the cells are selected for a particular property. For example, T cells that are CD4⁺ and CD8⁻ can be selected. B cells at various stages of maturity can be selected. In another example, the B cells are naive.

In one embodiment, fluorescent-activated cell sorting is used to sort B cells that express surface-bound IgM, IgD, or IgG molecules. Further, B cells expressing different isotypes of IgG can be isolated. In another preferred embodiment, the B or T cell is cultured in vitro. The cells can be stimulated in vitro, e.g., by culturing with feeder cells or by adding mitogens or other modulatory reagents, such as antibodies to CD40, CD40 ligand or CD20, phorbol myristate acetate, bacterial lipopolysaccharide, concanavalin A, phytohemagglutinin or pokeweed mitogen.

In still another embodiment, the cells are isolated from a subject that has an immunological disorder, e.g., systemic lupus erythematosus (SLE), rheumatoid arthritis, vasculitis, Sjogren syndrome, systemic sclerosis, or anti-phospholipid syndrome. The subject can be a human, or an animal, e.g., an animal model for the human disease, or an animal having an analogous disorder. In yet another embodiment, the cells are isolated from a transgenic non-human animal that includes a human immunoglobulin locus.

In one preferred embodiment, the cells have activated a program of somatic hypermutation. Cells can be stimulated to undergo somatic mutagenesis of immunoglobulin genes, for example, by treatment with anti-immunoglobulin, anti-CD40, and anti-CD38 antibodies (see, e.g., Bergthorsdottir et al. (2001) J Immunol. 166:2228). In another embodiment, the cells are naive.

Nucleic acids are prepared from these immune cells and are manipulated into a format for protein display.

Another type of naturally diversity is the diversity of sequences among different species of organisms. For example, diverse nucleic acid sequences can be amplified from environmental samples, such as soil and so forth.

Composite Libraries

A composite display library is assembled by pooling separately constructed display libraries, termed “component libraries” or “sublibraries” herein. The component libraries can include natural or synthetic diversity. A member isolated from the composite library can be identified as originating from one of the component libraries. This identification can be encoded in the nucleic acid sequence of the library member in one or both of two methods.

For the first method, information about the component library is encoded in a region that is constant among members of the component library. Corresponding positions in the other component libraries are designed to differ. The region that is constant can be a codon for a constant amino acid. At nucleic acid positions that encode constant amino acids, a single codon is used for each component library. The combination of codon use at the constant positions in any component library is designed to differentiate the component library from other component libraries. In implementations where only constant regions are used to identify the component libraries, then the combination of used codons should uniquely identify the component library.

Table 1 illustrates the nucleic acid sequence at two constant positions that are constrained to be cysteine. Cysteine can be encoded by one of two codons: TGT or TGC. These two cysteine positions are sufficient to differentiate four component libraries.

TABLE 1

| Component | Sequence | Sequence |
|---|---|---|
| Library | encoding Cys1 | encoding Cys2 |
| #1 | TGT | TGT |
| #2 | TGT | TGC |
| #3 | TGC | TGT |
| #4 | TGC | TGC |

In the second method, positions that vary within the component library are designed to provide information indicative of the source component library. For each position that varies, only a subset of codons for a particular amino acid are allowed. Ideally, only one codon is allowed for any amino acid that can appear at the position. The trinucleotide addition technology described above can be used to constrain the available codons at a given position while still allowing variation between encoded amino acids at that position.

Table 2 illustrates an example of how codons are constrained in at positions that vary in a library. At the first position, the encoded amino acid sequence is allowed to vary between Asn (encoded by AAT or AAC) and Gln (encoded by CAA or CAG). At the second position, the encoded amino acid sequence is allowed to vary between Arg (encoded by AGA, AGG, CGT, and three other codons not used here) and Lys (encoded by AAA or AAG).

TABLE 2

| Component | Sequence encoding | Sequence encoding |
|---|---|---|
| Library | Asn or Gln | Arg or Lys |
| #1 | AAT or CAA | AGA or AAA |
| #2 | AAT or CAA | AGG or AAG |
| #3 | AAC or CAG | AGA or AAA |
| #4 | AAC or CAG | AGG or AAG |
| #5 | AAT or CAA | AGA or AAA |
| #6 | AAT or CAA | AGG or AAG |
| #7 | AAT or CAA | CGT or AAA |
| #8 | AAC or CAG | AGA or AAA |
| #9 | AAC or CAG | AGG or AAG |
| #10 | AAC or CAG | CGT or AAA |

As shown in Table 2, a library member is selected from a composite library that includes libraries #1,2, 3, and 4. The library member from this composite library that includes AAC at the first position and AGA at the second position necessarily originates from library #3.

In another example, the assignment is ambiguous, but nevertheless reduces the possible number of originating component libraries. Such an assignment is still useful. In this example the composite library is constructed from component libraries #5, 6, 7, 8, 9, and 10. A library member from this composite library that has AAC and AGA at the first and second positions necessarily originates from library #8. However, a library member that has AAC and AAA at the first and second positions may have originated from either library #8 or #10.

One purpose for distinguishing among component libraries of a composite library is to for quality control. After display library members from a composite library are analyzed and sequenced, the originating component library for each library member is determined. Then, the number of useful identified display library members can be counted for each component library. Also, the frequency of insertions and deletions can be estimated for each component library. These statistics can be used to identify sub-optimal component libraries. Such libraries can be omitted from subsequently poolings for composite libraries.

Maturation Libraries

In one embodiment, display library technology is used in an iterative mode. A first display library is used to identify one or more ligands for a target. These identified ligands are then mutated to form a second display library. Higher affinity ligands are then selected from the second library, e.g., by using higher stringency or more competitive binding and washing conditions.

Numerous techniques can be used to mutate the identified ligands. These techniques include: error-prone PCR (Leung et al. (1989) Technique 1:11-15), recombination, DNA shuffling using random cleavage (Stemmer (1994) Nature 389-391; termed “nucleic acid shuffling”), RACHITT™ (Coco et al. (2001) Nature Biotech. 19:354), site-directed mutagenesis (Zooler et al. (1987) Nucl Acids Res 10:6487-6504), cassette mutagenesis (Reidhaar-Olson (1991) Methods Enzymol. 208:564-586) and incorporation of degenerate oligonucleotides (Griffiths et al. (1994) EMBO J 13:3245).

If, for example, the identified ligands are antibodies, then mutagenesis can be directed to the CDR regions of the heavy or light chains. Further, mutagenesis can be directed to framework regions near or adjacent to the CDRs. Likewise, if the identified ligands are enzymes, mutagenesis can be directed to the vicinity of the active site.

Negative Selection

The display library screening methods described herein can also include a selection step that removes display library members that bind to a non-target molecules. This so-called “negative selection” can be used to identify display library members that discriminate between a target molecule and a related, but distinct non-target molecule. In the case of polypeptide targets and nucleic acid targets, the non-target and the target molecules can be at least 30%, 50%, 75%, 80%, 90%, 95%, 98%, or 99% identical to each other. They can differ only in a small region which is the intended epitope for recognition. The non-target and target molecule can be identical, but can have different conformations, oligomerization states, or modifications (e.g., a post-translational modification for polypeptide; a methylation or base adduct for nucleic acid). In one embodiment, the target is a complex of at least two polypeptides, and the non-targets are the component polypeptides in their uncomplexed state. An illustrative case is one in which the target is fibrin, and the non-target is fibrinogen. Fibrin is a processed form of fibrinogen that forms a mesh structure that includes epitopes absent from fibrinogen although all amino acids of fibrin are present in the fibrinogen sequence. In still another embodiment, the non-target is a constant region, e.g., a peptide tag, purification handle, or attachment moiety that is present during the selection of the target molecule.

In another example, the non-target and target molecules are at least 30%, 50%, 60%, 70%, or 80% divergent.

The display library or a pool thereof is first contacted to the non-target molecule. Members of the sample that do not bind the non-target can be collected and used in subsequent selections for binding to the target molecule or even for subsequent negative selections. This procedure aids the identification of display library members that bind to the target, but not the non-target.

Off-Rate Selection

Since a slow dissociation rate can be predictive of high affinity, particularly with respect to interactions between polypeptides and their targets, methods can be used to isolate biomolecules with a selected kinetic dissociation rate for a binding interaction to an immobilized target. An off-rate selection includes binding members of a display library to a target, and washing the target of non-specifically and weakly bound members. Then, the immobilized target is contacted with an elution solution that includes a saturation amount of free target, i.e., replicates of the target that are not immobilized. The free target binds to display library members that dissociate from the immobilized target molecules. Rebinding is effectively prevented by the saturating amount of free target relative to the much lower concentration of target attached to the particles.

The elution solution is collected at regular intervals. Display library members that are eluted during later intervals are likely to have a slower dissociation rate than those members that elute in earlier intervals. Further, display library members that are cannot be eluted from the target can also be recovered. For example, if the target is bound to a support, the target itself can be separated from the support. In another example, the display library member or its nucleic acid component is recovered directly from the support.

The automated selection apparati described herein, e.g., magnetic particle processors, can be programmed for off-rate selection. For example, the target is immobilized on magnetic particles and moved into tubes include the elution solution at time intervals that separate library members that dissociate early from members that dissociate later.

Targets

Generally, any molecular species can be used as a target. The target can be of a small molecule (e.g., a small organic or inorganic molecule), a polypeptide, a nucleic acid, cells, and so forth. By way of example, a number of examples and configurations are described for targets. Of course, targets other than, or having properties other, than those listed below can also be used.

One class of targets includes polypeptides. Examples of such targets include small peptides (e.g., about 3 to 30 amino acids in length), single polypeptide chains, and multimeric polypeptides (e.g., protein complexes).

A polypeptide target can be modified, e.g., glycosylated, phosphorylated, ubiquitinated, methylated, cleaved, disulfide bonded and so forth. Preferably, the polypeptide has a specific conformation, e.g., a native state or a non-native state. In one embodiment, the polypeptide has more than one specific conformation. For example, prions can adopt more than one conformation. Either the native or the diseased conformation can be a desirable target, e.g., to isolate agents that stabilize the native conformation or that identify or target the diseased conformation.

In some cases, however, the polypeptide is unstructured, e.g., adopts a random coil conformation or lacks a single stable conformation. Agents that bind to an unstructured polypeptide can be used to identify the polypeptide when it is denatured, e.g., in a denaturing SDS-PAGE gel, or to separate unstructured isoforms of the polypeptide for correctly folded isoforms, e.g., in a preparative purification process.

Some exemplary polypeptide targets include: cell surface proteins (e.g., glycosylated surface proteins or hypoglycosylated variants), cancer-associated proteins, cytokines, chemokines, peptide hormones, neurotransmitters, cell surface receptors (e.g., cell surface receptor kinases, seven transmembrane receptors, virus receptors and co-receptors, extracellular matrix binding proteins, or a cell surface protein (e.g., of a mammalian cancer cell or a pathogen). In some embodiments, the polypeptide is associated with a disease, e.g., cancer.

More specific examples include: integrins, cell attachment molecules or “CAMs” such as cadherins, selections, N-CAM, E-CAM, U-CAM, I-CAM and so forth); proteases, e.g., subtilisin, trypsin, chymotrypsin; a plasminogen activator, such as urokinase or human tissue-type plasminogen activator (t-PA); bombesin; factor IX, thrombin; CD-4; CD-19; CD20; platelet-derived growth factor; insulin-like growth factor-I and -II; nerve growth factor; fibroblast growth factor (e.g., aFGF and bFGF); epidermal growth factor (EGF); transforming growth factor (TGF, e.g., TGF-α and TGF-β); insulin-like growth factor binding proteins; erythropoietin; thrombopoietin; mucins; human serum albumin; growth hormone (e.g., human growth hormone); proinsulin, insulin A-chain insulin B-chain; parathyroid hormone; thyroid stimulating hormone; thyroxine; follicle stimulating hormone; calcitonin; atrial natriuretic peptides A, B or C; leutinizing hormone; glucagon; factor VIII; hemopoietic growth factor; tumor necrosis factor (e.g., TNF-α and TNF-β); enkephalinase; mullerian-inhibiting substance; gonadotropin-associated peptide;; tissue factor protein; inhibin; activin; vascular endothelial growth factor; receptors for hormones or growth factors; protein A or D; rheumatoid factors; osteoinductive factors; an interferon, e.g., interferon-α,β,γ; colony stimulating factors (CSFs), e.g., M-CSF, GM-CSF, and G-CSF; interleukins (ILs), e.g., IL-1, IL-2, IL-3, IL-4, etc.; decay accelerating factor; immunoglobulin (constant or variable domains); and fragments of any of the above-listed polypeptides. In some embodiments, the target is associated with a disease, e.g., cancer.

The target polypeptide is preferably soluble. For example, soluble domains or fragments of a protein can be used. This option is particularly useful for identifying molecules that bind to transmembrane proteins such as cell surface receptors and retroviral surface proteins.

Another class of targets includes cells, e.g., fixed or living cells. The cell can be bound to an antibody that is covalently attached to a paramagnetic particle or indirectly attached (e.g., via another antibody). For example, a biotinylated rabbit anti-mouse Ig antibody is bound to streptavidin paramagnetic beads and a mouse antibody specific for a cell surface protein of interest is bound to the rabbit antibody.

In one embodiment, the cell is a recombinant cell, e.g., a cell transformed with a heterologous nucleic acid that expresses a heterologous gene or that disrupts or alters expression of an endogenous gene. The heterologous nucleic acid can be under control of an inducible or constitutive promoter. In a preferred embodiment, the heterologous nucleic acid encodes a cell surface protein, e.g., a cell-surface protein of interest. The plasmid can also express a marker protein, e.g., for use in binding the transformed cell to a magnetically responsive particle.

In another embodiment, the cell is a primary culture cell isolated from a subject, e.g., a patient, e.g., a cancer patient. In still another embodiment, the cell is a transformed cell, e.g., a mammalian cell with a cell proliferative disorder, e.g., a neoplastic disorder. In still another embodiment, the cell is the cell of a pathogen, e.g., a microorganism such as a pathogenic bacterium, pathogenic fungus, or a pathogenic protist (e.g., a Plasmodium cell) or a cell derived from a multicellular pathogen. The target can also be a cell, e.g., a cancer cell, a hematopoietic cell, , and so forth.

In still another embodiment, the cells are treated (e.g., using a drug or genetic alteration). For example, the treatment can alter the rate of endocytosis, pinocytosis, exocytosis, and/or cell secretion. The treatment can also be a drug or an inducer of a heterologous promoter-subject gene construct. The treatment can cause a change in cell behavior, morphology, and so forth. Molecules that dissociate from the cells upon treatment or that associate with cells when treated are collected and analyzed.

In another embodiment, the target is a tissue or organ. The display library can be screened for members that bind to the tissue or organ in vitro or in vivo (e.g., as described in Kolonin et al. (2001) Current Opinion in Chemical Biology 5:308-313).

Additional exemplary targets include nucleic acids, e.g., double-stranded, single-stranded, and partially double-stranded DNA such as a site in a regulatory region, a site in a coding region, a tertiary structure e.g., a G-quartet or a telomere; RNA, e.g., double-stranded RNA, single-stranded RNA, e.g., an RNAi, a ribozyme; or combinations thereof. For example, a double stranded nucleic acid that includes a site can be used to identify a DNA-binding domain that binds to that site. The DNA-binding domain can be used in cells to regulate genes that are operably linked to the site. For example, the methods described herein can be used to screen a library of zinc finger polypeptides for binding to a target nucleic acid. See, e.g., Rebar et al. (1996) Methods Enzymol. 267:129-49. No abstract available for a description of phage display libraries of zinc finger polypeptides.

Still more exemplary targets include organic molecules. In one embodiment, the organic molecules are transition state analogues and can be used to select for catalysts that stabilize a transition state structure similar to the structure of the analogue. In another embodiment, the organic molecules are suicide substrates that covalently attach to catalysts as a result of the catalyzed reaction.

A target can be a drug, e.g., a drug for which a ligand is required in order to improve purification of the drug, e.g., from a chemical reaction, a bioreactor, a media, milk, or a cell extract. The drug can include a peptide, e.g., a polypeptide or a non-peptide functionality.

Other targets may be relevant to biotechnological applications, e.g., to generate molecules useful for the laboratory. For example, streptavidin, green fluorescent protein, or a nucleic acid polymerase can be a target.

In some embodiments, more than one species is used as a target, e.g., a sample is exposed to a plurality of targets.

Therapeutic Uses

The screening methods described herein can be used to identify a protein with therapeutic properties. The protein can be used, e.g., for treatment, prophylaxis, general improvement with respect to a condition. The protein can be formulated with a pharmaceutically acceptable carrier to provide a pharmaceutical composition.

In another aspect, the present invention provides compositions, which include a target-specific ligand, e.g., an antibody molecule, other polypeptide or peptide identified as binding to a target molecule using the method described herein, formulated together with a pharmaceutically acceptable carrier. Pharmaceutical compositions can encompass labeled ligands for in vivo imaging as well as therapeutic compositions.

As used herein, “pharmaceutically acceptable carriers” include any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., protein ligand may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for administration of humans with antibodies. The preferred mode of administration is parenteral (e.g., intravenous, subcutaneous, intraperitoneal, intramuscular). In a preferred embodiment, the target-specific ligand is administered by intravenous infusion or injection. For example, for therapeutic applications, the target-specific ligand can be administered by intravenous infusion at a rate of less than 30, 20, 10, 5, or 1 mg/min to reach a dose of about 1 to 100 mg/m² or 7 to 25 mg/m². The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

In certain embodiments, the ligand may be orally administered, for example, with an inert diluent or an assimilable edible carrier. Pharmaceutical compositions can be administered with medical devices known in the art.

Diagnostic Uses

Proteins identified by the screening methods described herein can be used to detect the target compound to which they bind, e.g., for detecting the presence of the target, in vitro (e.g., a biological sample, such as tissue, biopsy, e.g., a cancerous tissue) or in vivo (e.g., in vivo imaging in a subject). The following are merely exemplary uses of a target-specific ligand. These include: ELISA assays, FACS analysis and sorting, microscopy, protein arrays, and in vivo imaging. These applications can be performed for one target-specific ligand, or in a high-thoughput mode for many

A target specific ligand can be labeled, e.g., using fluorophore and chromophore labeled protein ligands. Since antibodies and other proteins absorb light having wavelengths up to about 310 nm, the fluorescent moieties should be selected to have substantial absorption at wavelengths above 310 nm and preferably above 400 nm. A variety of suitable fluorescers and chromophores are described by Stryer (1968) Science, 162:526 and Brand, L. et al. (1972) Annual Review of Biochemistry, 41:843-868. The protein ligands can be labeled with fluorescent chromophore groups by conventional procedures such as those disclosed in U.S. Pat. Nos. 3,940,475, 4,289,747, and 4,376,110. One group of fluorescers having a number of the desirable properties described above is the xanthene dyes, which include the fluoresceins and rhodamines. Another group of fluorescent compounds are the naphthylamines. Once labeled with a fluorophore or chromophore, the protein ligand can be used to detect the presence or localization of the target molecule in a sample, e.g., using fluorescent microscopy (such as confocal or deconvolution microscopy).

Histological Analysis. Immunohistochemistry can be performed using the target-specific ligands identified by the methods described herein. The ligand is labeled, and contacted to a histological preparation, e.g., a fixed section of tissue that is on a microscope slide. After an incubation for binding, the preparation is washed to remove unbound antibody. The preparation is then analyzed, e.g., using microscopy, to identify if the ligand bound to the preparation.

Protein Arrays. A target-specific ligand identified by a method described herein can be immobilized on a protein array. The protein array can be used as a diagnostic tool, e.g., to screen medical samples (such as isolated cells, blood, sera, biopsies, and the like). Methods of producing polypeptide arrays are described, e.g., in De Wildt et al. (2000) Nat. Biotechnol. 18:989-994; Lueking et al. (1999) Anal. Biochem. 270:103-111; Ge (2000) Nucleic Acids Res. 28, e3, I-VII; MacBeath and Schreiber (2000) Science 289:1760-1763; WO 01/40803 and WO 99/51773A1. Polypeptides for the array can be spotted at high speed, e.g., using commercially available robotic apparati, e.g., from Genetic MicroSystems or BioRobotics. The array substrate can be, for example, nitrocellulose, plastic, glass, e.g., surface-modified glass. The array can also include a porous matrix, e.g., acrylamide, agarose, or another polymer.

In vivo Imaging. In still another embodiment, the target-specific ligands identified by the methods herein are conjugated to a detectable marker, administered to a subject, and imaged by detecting the detectable marker bound to tareget-expressing tissues or cells. For example, the subject is imaged, e.g., by NMR or other tomographic means.

Examples of labels useful for diagnostic imaging in accordance with the present invention include radiolabels such as ¹³¹I, ¹¹¹In, ¹²³I, ^{99m}Tc, ³²P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed. The protein ligand can be labeled with such reagents using known techniques. For example, see Wensel and Meares (1983) Radioimmunoimaging and Radioimmunotherapy, Elsevier, New York for techniques relating to the radiolabeling of antibodies and D. Colcher et al. (1986) Meth. Enzymol. 121: 802-816. NMR signals can be enhanced by contrast agents. Examples of such contrast agents include a number of magnetic agents paramagnetic agents (which primarily alter T1) and ferromagnetic or superparamagnetic (which primarily alter T2 response). The target-specific ligands can also be labeled with an indicating group containing of the NMR-active ¹⁹F atom. After permitting time for target binding, a whole body MRI is carried out using an apparatus such as one of those described by Pykett (1982) Scientific American, 246:78-88 to locate and image cancerous tissues.

Purification Uses

Proteins identified by the screening methods described herein can be used to purify the target compounds. In one embodiment, the purification is on a production scale, e.g., to purify a protein pharmaceutical or other pharmaceutical. A target-specific ligand identified by the methods herein can be couple to a support and used as an affinity reagent in affinity chromatography. Scopes (1994) Protein Purification: Principles and Practice, New York:Springer-Verlag provides a number of methods for purifying recombinant and non-recombinant proteins by affinity chromatography. The use of a customized target specific ligand can obviate the need for an affinity tag, and/or can enable highly specific separation of closely related isoforms. See, e.g., U.S. Pat. No. 6,326,155.

Additional Exemplary Libraries

Other types of libraries for which aspects of this disclosure can be implemented include a protein expression library (e.g., a cDNA library, e.g., for a cellular phenotype, intracellular expression), a two-hybrid library, a protein array, a nucleic aptamer, a chemical library such as combinatorial library or a drug compound library.

Nucleic acid libraries, generally. Library construction methods described herein can include use of routine techniques in the field of molecular biology, biochemistry, classical genetics, and recombinant genetics. Basic texts disclosing the general methods of use in this invention include Sambrook et al., Molecular Cloning, A Laboratory Manual (2nd ed. 1989); Kriegler, Gene Transfer and Expression:A Laboratory Manual (1990); and Current Protocols in Molecular Biology (Ausubel et al., eds., 1994)).

To make a cDNA library, one can choose a source that is rich in the RNA of choice. The mRNA is then made into cDNA using reverse transcriptase, ligated into a recombinant vector, and transfixed into a recombinant host for propagation, screening and cloning. Methods for making and screening cDNA libraries are well known (see, e.g., Gubler & Hoffman, Gene 25:263-269 (1983); Sambrook et al., supra; Ausubel et al., supra). Exemplary methods for screening cDNA libraries include: U.S. Pat. Nos. 5,866,098 and 5,654,150.

For a genomic library, the DNA is extracted from the tissue and either mechanically sheared or enzymatically digested to yield fragments of about 12-20 kb. The fragments are then separated by gradient centrifugation from undesired sizes and are constructed in eukaryotic plasmid vectors, yeast artificial chromosomes, P1s, or bacteriophage lambda vectors. Phage vectors are packaged in vitro.

Two-Hybrid. A two-hybrid assay or three-hybrid assay can be used to screen libraries of proteins to identify interacting proteins (or RNA-protein interactions). See, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300. The two-hybrid system is based on the modular nature of most transcription factors, which consist of separable DNA-binding and activation domains. Briefly, the assay utilizes two different DNA constructs. In one construct, the gene that codes for a protein of interest is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the protein of interest.

Nucleic acid aptamers. Nucleic acid aptamer libraries are pools of diverse nucleic acid sequences from which nucleic acids are selected for binding or catalytic properties that are conferred by the nucleic acid molecules themselves. Random pools of nucleic acid sequences, both DNA and RNA, can be used as a rich source of artificial ligands and catalysts (see, e.g., Ellington and Szostak (1990) Nature 346:818; and (1992) Nature 355:850; and Tuerk and Gold ((1990) Science 249:505 and (1991) J. Mol. Biol. 222:739; U.S. Pat. No. 5,910,408). Such artificial nucleic acid are termed aptamers. Generally, synthetic oligonucleotides are used to assemble pools of random nucleic acid sequences. The sequences can include a constant region or tag which can serve as a primer binding site. The pools are exposed to the target, which can be an intended ligand or a transition state analog. Nucleic acids in the pool that bind the target are selected and then either pooled for subsequent selections after nucleic acid amplification, or cloned into a vector. Nucleic acid aptamers that are cloned into a vector are transformed into a host cell and plated. Individual clones can then be processed using the methods described above. Replicates of the nucleic acid in each individual clone can be recovered by amplifying the clone nucleic acid with the appropriate primers, and if necessary rendering it single stranded.

Other Chemical Libraries. Examples combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)); peptoids (e.g., PCT Publication No. WO 91/19735); benzodiazepines (e.g., U.S. Pat. No. 5,288,514); diversomers such as hydantoins, benzodiazepines and dipeptides (Hobbs et al., Proc. Nat. Acad. Sci. USA 90:6909-6913 (1993)); oligocarbamates (Cho et al., Science 261:1303 (1993)); carbohydrate libraries (see, e.g., Liang et al., Science, 274:1520-1522 (1996) and U.S. Pat. No. 5,593,853); and other small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. Nos. 5,506,337; benzodiazepines, 5,288,514, and the like).

It is to be understood that while the invention has been described in conjunction with the detailed description thereof, the foregoing description is intended to illustrate and not limit the scope of the invention, which is defined by the scope of the appended claims. For example, aspects of the invention are applicable to implementations using nucleic acid expression libraries (e.g., cDNA expression libraries), nucleic acid aptamer libraries, combinatorial chemical libraries, and synthetic peptide libraries. Other embodiments are within the following claims.

What is claimed:

1. A machine-based method for managing library information, the method comprising:
storing information that comprises associations between (a) individual library members and (b) assay information about each of a plurality of the individual library members; and
evaluating the stored information to identify a subset of the individual library members.

2. The method of claim 1 wherein the library members comprise display library members.

3. The method of claim 2 wherein the evaluating comprises filtering the stored information to identify a subset of display library members for which the associated assay data meets a criterion.

4. The method of claim 3 further comprising, prior to the filtering, receiving a query that comprises information about the criterion.

5. The method of claim 2 in which the display library members comprise members that are isolated from the library in a first selection and members that isolated from a library in a second selection.

6. The method of claim 2 wherein the display library members are identified by physical location of a clone of each respective display library member.

7. The method of claim 1 in which the assay data relates to an in vitro assessment of activity.

8. The method of claim 7 in which the activity is a binding activity.

9. The method of claim 2 in which each of the display library members are stored at a unique address of one or more first arrays, and the method further comprises instructing a sample handling instrument to transfer each member of the subset to a unique address of one or more second arrays such that the order or total number of stored library members differs from the order of total number stored in the first array.

10. A system comprising:
a display console that includes a graphical user interface;
a communications interface that sends and receives information to a laboratory apparatus; and
a processor configured to execute a method comprising:
receiving information from the communications interface, the information comprising library member assay data;
storing information that comprises associations between (a) library members and (b) the library member assay data;
evaluating the stored information to identify a subset of library members; and
display results of the evaluating on the display console.

11. A method of selecting a library member, the method comprising:
providing a library that comprises a plurality of members, each member of the plurality including a nucleic acid that encodes a diverse protein component;
selecting a set of members from the library;
evaluating the set of the members to obtain a functional parameter for each respective member of the set;
sending information about the functional parameter for each respective member of the set to a computer server for storage;
querying the server with a criterion for functionality that can be used to identify a subset of the set that are characterized by functional parameter that satisfies the criterion; and
filtering the stored information to identify a subset of library members for which the associated functional assay data meets the criterion.

12. The method of claim 11 wherein each member of the plurality further includes the diverse protein component encoded by the nucleic acid of the respective member.

13. The method of claim 12 wherein selecting the set of library members comprises contacting the display library members to a target; and separating members that bind to the target from other members that do not bind to the target.

14. The method of claim 12 wherein selecting the set of library members comprises fewer than four cycles of: (a) contacting the library members to a target; (b) separating members that bind to the target from other members that do not bind to the target, and, optionally, (c) amplifying members that bind to the target.

15. The method of claim 11 further comprising:
producing a protein corresponding to the diverse protein component of a member of the identified subset, and formulating the protein as a pharmaceutical composition.

16. A machine-accessible medium which comprises:
data representing (a) information about screens to identify a polypeptide having a given property, (b) identifiers for display library members that each encode a polypeptide, (c) results of functional assays for at least some of the display library members; (d) biopolymer sequences for at least some of the display library members; and
associations that relate (1) the screen information and the display library member identifiers; and (2) display library member identifiers and functional assay results.

17. The medium of claim 16 wherein the information about screens includes information about one or more of: a target, a screening condition, and a library.

18. The medium of claim 16 wherein the data further represents information about projects, each project being associated with information about one or more screens.

19. The medium of claim 18 wherein the information about each of at least some of the projects is further associated with information about a client.

20. The medium of claim 19 further comprising data representing information about clients and billing.

21. A system comprising:
a nucleic acid sequencing instrument that is configured to determine the nucleic acid sequence of display library members selected by a display library screen;
an assay apparatus that is configured to assess a functional property of the selected display library members; and
a server comprising:
a communication interface that receives information about the determined nucleic acid sequence of each of the selected display library members from the nucleic acid sequence instrument and information about the assessed functional property for each of the selected display library members from the assay apparatus,
a memory that stores the received information in association with information about the display library screen, and
a processor that filters the received information to identify a subset of the select display library members.

22. The system of claim 21 further comprising a sample picking apparatus configured to dispose picked samples into wells of a multiwell plate.

23. The system of claim 21 in which the sample picking apparatus comprises a detector that detects a multiwell plate identifier on the multiwell plate and the sample picking apparatus is interfaced with the server to communicate information about the detected multiwell plate identifiers to the server.

24. The system of claim 21 in which the system generates an automatic alert.

25. A system comprising:
a server storing (i) information about the determined nucleic acid sequence of each of a plurality of selected display library members from the nucleic acid sequence instrument and information about the assessed functional property for each of the display library members from the assay apparatus, and (ii) software configured to receive a query, filter the stored information to identify a subset of the selected display library members, and distribute information about the subset of the selected display library members.

26. A method comprising:
automatically receiving, from a nucleic acid sequencer, information about the nucleic acid sequence of library members identified in one or more library screens;
automatically receiving, from an assay device, information about functionality of the library members;
storing the received information in association with identifiers for the library members and an identifier for the library screen.

27. The method of claim 26 wherein the library members are member of a display library.

28. The method of claim 26 wherein the assay device detects a sample identifier on a multiwell plate that includes samples of the library member and the information received from the assay device includes information about the sample identifier.

29. A method of evaluating display library members, the method comprising:
receiving information representing a plurality of biological sequences, each sequence corresponding to a display library member selected from a display library;
evaluating the information for each biological sequence of the plurality to determine the location of a sequence feature, if present, wherein the sequence feature is characteristic of at least a plurality of members of the display library; and
storing or displaying information for a subsequence from each biological sequence for which the sequence feature is identified, the subsequence being defined as a function of the position of the sequence feature.

30. The method of claim 29 wherein each member of the display library displays a protein comprising an immunoglobulin variable domain, and the display library includes at least 10 different sequence variants of the immunoglobulin variable domain.

31. A method of evaluating a display library, the method comprising:
disposing display library members into a first set of multiwell plates, each display library member being picked into a unique well of one of the multiwell plates of the first set;
amplifying each display library member;
determining an assessment for each display library member with respect to a property;
storing information about the assessments of the display library members; and
filtering the information to identify a subset of the display library members.

32. The method of claim 31 further comprising manipulating each display library member of the subset into a second set of multiwell plates.

33. The method of claim 31 further comprising, prior to the picking, selecting the display library members for binding to a target using a magnetic particle processor.

34. A method of managing events associated with screening a library, the method comprising:
accessing stored information that comprises event identifiers, at least some of the identifiers being associated with a first screening of a library;
receiving a request for an event identifier for an event that relates to a second screening of a library; and
generating an event identifier unique among the event identifiers in the stored information, wherein the first and second screening are library screens for proteins that have a first and second property, respectively.

35. A method of handling event information for library screening, the method comprising:
receiving, from a first workstation, information about a first event associated with a first screening of a library;
receiving, from a second workstation, information about a second event associated with the first screening; and
storing the information about the first event and information about the second event in association with an indication of the first screening or other information associated with the first screening.

36. The method of claim 35 further comprising labelling a multi-well plate with the unique event identifier.

37. The method of claim 36 further comprising tracking the multi-well plate.

38. The method of claim 36 wherein the unique event identifier is labeled as a optically-detectable code.

39. A machine-based method of managing a display library project, the method comprising:
initializing a project identifier for a project;
generating a unique container identifier that is associated with the project identifier for a first multi-well container of display library members;
labelling the multi-well container with the unique container identifier; and
automatically disposing individual display library members into wells of the multiwell container.

40. A method of evaluating a member of a composite nucleic acid library, the method comprising:
receiving information about a nucleic acid sequence of a library member that is isolated from a composite of at least two libraries, wherein each library of the composite is constructed using a different limited set of codons at at least one position;
parsing the information about the nucleic acid sequence into codons; and
identifying an originating library from the libraries of the composite on the basis of the codon of the nucleic acid sequence at the at least one position.

41. A method of providing a composite nucleic acid library, the method comprising:
constructing a first library of nucleic acids wherein each member of the first library includes one of a limited set of codons at at least one varying position;
constructing a second library of nucleic acids wherein each member of the second library includes one of a limited set of codons at at least one varying position; and
pooling members of the first and second library, thereby providing a composite nucleic acid library.

42. The method of claim 41, wherein the limited set of codons of nucleic acids of the first library differs from the limited set of codons of nucleic acids of the second library at at least one corresponding varying position.

43. The method of claim 41, wherein the codon usage at at least one corresponding constant position differs between nucleic acids of the first and second libraries.

44. A user interface that enables a user to access the medium of claim 16; select a subset of display library members and displays information about each member of the subset.

45. The user interface of claim 44 that further enables the user to distribute the displayed information to other users.

46. A method of screening a display library, the method comprising:
providing a display library that comprises a plurality of members, each member including a diverse protein component and a nucleic acid that encodes the diverse protein component;
selecting a set of members from the display library using one or more cycles of binding to a target and separation; and
processing members of the selected set using the system of claim 21.

47. The user interface of claim 44 wherein the individual display library members are identified in screens using different targets.

48. A server configured to:
store information about display library screens and display library members identified in the screens,
authenticate a remote user for permission to access the stored information for a subset of the display library screens;
receive queries from the remote user for information about display library members that satisfy a criterion;
filter the stored information to identify selected library members that are identified in the subset of the display library screens and that satisfy the criterion; and
send to the remote user information about the selected library members.

49. The method of claim 48 wherein each of the screens is associated with a client, and the remote user is authenticated if the remote user is identified as the client.

50. An article of machine readable medium having instructions encoded thereon, the instructions causing a processor to effect the method of claim 1.

51. The system of claim 10 wherein the library members comprise display library members.

52. The method of claim 35 wherein the library is a display library.

53. The method of claim 34 wherein the library used for the first screening and the library for the second screening are display libraries.

54. The method of claim 34 wherein the first and second property are ability to interact with a first and second target, respectively.
